(19) United States
(12) Patent Application Publication
(10) Pub. No.: US 2003/0147973 A1
(43) Pub. Date: Aug. 7, 2003

(54) Use of extracts from aristolochia in the treatment of aids

(76) Inventors: Hans Achenbach, Wiesbaden (DE); Carlos Navarro, Houston, TX (US); Alejandro Navarro, Juarez Chihuahua (MX)

Correspondence Address: BAKER & BOTTS, 30 ROCKEFELLER PLAZA, NEW YORK, NY, 10112

(21) Appl. No.: 10/322,305

(22) Filed: Dec. 18, 2002

Related U.S. Application Data
(63) Continuation of application No. 09/774,769, filed on Jan. 31, 2001, now abandoned.
Application No. 09/774,769 is a continuation of application No. PCT/US99/02194, filed on Feb. 2, 1999.

(30) Foreign Application Priority Data
Jul. 31, 1998 (WO) PCT/GB98/02317

(51) Int. Cl. A61K 35/78

(52) U.S. Cl. 424/725

(57) ABSTRACT

This invention relates to the use of extracts and compounds derived from the plant Aristolochia taliscana and their analogues in the treatment of AIDS.

### FIELD OF THE INVENTION

This invention relates to compounds derived from the plant Aristolochia taliscana and their analogues, and the uses of such compounds in medicine.

### BACKGROUND OF THE INVENTION

Aristolochia taliscana, a climbing shrub found in the jungles of the southern coastal region of Mexico, is part of a family of climbing herbs and shrubs called Aristolochiaceae, numbering about six hundred species divided into eleven genera, and found mostly in tropical and sub-tropical regions. It is believed that the species Aristolochia taliscana is found only in Mexico.

Members of the Aristolochiaceae are known for their ability to synthesise phenanthrene alkaloids, and in particular the aristolactam alkaloids and the aristolochic acids, and arylpropanoid compounds such as the lignans and neolignans. Such compounds are disclosed in, for example, R. Hegnauer “Chemotaxonomie der Pflanzen”, Vol. III, pp 184-199, Birkhäuser Verlag, Basel und Stuttgart, 1964; R. Hegnauer “Chemotaxonomie der pflanzen”, Vol. VII, pp 75-83, Birkhäuser Verlag, Basel-Boston-Berlin, 1989 and F. E. Correa et al. “Especies Vegetales Promisorios”, Vol. I, pp440-469, Secretaria Ejecutiva del Convenio Andies Bello (SECAB), Bogota D. E. 1989, Colombia and Lopes et al. Rev. Latinoam. Quim., 19 (3-4), 113-17, 1988. In Lopes et al., for example, the isolation of lignans from a number of different Aristolochiaceae is described and it is disclosed that such compounds are reported as having anti-tumour, antifungal, antibacterial and insecticidal activity. In Hinou et al., J. Crude Drug Research, 1990, 28(2), 149-51, it is disclosed that aristolactam and aristolochic acid compounds isolated from Aristolochia longa have antibacterial activity and cytotoxic activity against P-388 lymphocytic leukaemia and human bronchial epidermoid carcinoma cells.

The isolation and characterisation of lignans, neolignans and related compounds from a wide variety of plant species has been reviewed in a series of articles by R. S. Ward, see for example Natural Product Reports, 1985, Vol. 5 pp203-206; 1990, Vol. 7, pp356-363; 1993, Vol. 10, pp1-23.

However, it is clear from the available literature that the chemical structures and concentrations of arylpropanoid compounds found in Aristolochiaceae vary widely from one species to another. For example, in Lopes et al. (idem.), reference is made to the extraction of four Brazilian species of Aristolochiaceae, from which a number of dibenzyl-butyrolactone type lignans and furofuran type lignans were isolated. From studies made by the present inventors, such compounds would appear to be absent from Aristolochia taliscana.

Much of the work carried out on the Aristolochiaceae has focused on the phenanthrene alkaloid content, and in particular the aristolactam alkaloids found in the plants—see for example Crohare et al. Phytochemistry, 1974, Vol. 13, 1957-1962, Priestap, Phytochemistry, 1985, Vol. 24, 849-852, Talapatra et al. Phytochemistry, 1988, Vol. 27, 903-906 and Houghton et al. Phytochemistry, 1991, Vol. 10, 253-254. Houghton et al. suggest that compounds such as aristolochic acid, the ring-opened form of aristolactam, are of interest as immunostimulants and anticancer agents.

Crude extracts from Aristolochia taliscana have been known for many years to have certain medicinal properties. A book published in the 1800's, called “Las Plantas Medicinales de Mexico” (Medicinal Plants of Mexico) makes reference to the use of taliscanine in the treatment of snake bites and as a sexual stimulant, and it would appear that the native tribes in this region of Mexico have known about the uses of taliscanine for many centuries.

In U.S. Pat. No. 4,782,077 it is disclosed that taliscanine, an extract from the root of Aristolochia taliscana, alleviates the symptoms of Parkinsonism and related neurological disorders. It is also indicated in U.S. Pat. No. 4,782,077 that taliscanine may be useful in the treatment of various other neurological disorders, including Alzheimer's disease, impotency, and neurological disorders associated with viral, bacterial, fungal and parasitic infections.

In U.S. Pat. No. 4,782,077, the extract for which the foregoing activities were disclosed was prepared by pulverising Aristolochia taliscana root and subjecting the powder to soxhlet extraction with hexane and then benzene followed by column chromatography on an alumina column eluting with benzene-ether mixtures. The resulting compound was characterised as being the known aristolactam taliscanine, on the basis of its melting point (272°-273° C.) and its spectroscopic data.

However, taliscanine has since been tested for its ability to interact with neurotransmitter receptors, and, somewhat surprisingly, exhibited 50% inhibition in only one receptor (the opiate mu receptor) out of twenty seven common receptor types tested, and exhibited very poor levels of inhibition with the remaining receptors. In particular, taliscanine exhibited negligible activity at the dopamine, GABA and serotonin receptors. These results suggest either that taliscanine exerts its neurological effects by a mechanism which is of a currently unknown type (which seems unlikely) or, perhaps, that there is another active principle in Aristolochia taliscana which is responsible for the reported activities.

### SUMMARY OF THE INVENTION

The present applicants have now found that the administration of extracts of Aristolochia taliscana to patients suffering from acquired immune deficiency syndrome (AIDS) brings about a substantial improvement in the condition of such patients. In particular, extracts from Aristolochia taliscana have been found to prevent the appearance of the symptoms of AIDS, and to reverse existing symptoms of AIDS, for example cachexia. Such results have been observed even though there is seemingly no improvement in the immune system of the patient. For example, in one patient, the CD4 count fell to a value of 60, well below the figure of 200 which is generally recognised as being the threshold for the onset of full-blown AIDS. In another case, a patient suffering from cachexia as a result of AIDS has been found to regain weight and is otherwise asymptomatic.

Accordingly, in one aspect, the invention provides the use of an extract from an Aristolochia species, preferably Aristolochia taliscana, or one or more compounds isolable therefrom, for the manufacture of a medicament for the treatment of AIDS.

Additionally, the invention provides the use of an extract or compound as hereinbefore defined for the manufacture of a medicament for preventing or reversing cachexia, for example in AIDS patients, or in patients suffering from neoplastic diseases such as cancers.

The extracts from the Aristolochia species (e.g. Aristolochia taliscana) can be prepared by extracting the roots, stems, leaves or other parts of the plant with an organic solvent such as ethanol.

Extracts from Aristolochia taliscana have also been found to be useful in the treatment of male impotence. In a further aspect, therefore, the invention provides for the use of a compound or compounds isolable from Aristolochia taliscana for the manufacture of a medicament for the treatment of male impotence.

The present applicants have been able to separate and identify the components of taliscanine and have found that the extract contains a substantial number of compounds other than aristolactams, in particular certain benzofuran neolignans, many of which are novel. Benzofuran compounds isolated from taliscanine have been tested and have been found to be active as anti-mutagenic agents, as cytotoxic agents, and some have been found to have good antifungal activity. On this basis, it is anticipated that the compounds in question will find use in the treatment of tumours and other neoplastic diseases, as well as fungal infections.

Accordingly, in another aspect, the invention provides the use of an extract of Aristolochia taliscana or one or more anti-mutagenically active components isolable therefrom for the manufacture of a medicament for the treatment of disease states mediated by mutagenesis.

The invention also provides the use of an extract of an Aristolochia species, preferably Aristolochia taliscana or one or more component compounds isolable therefrom, for the manufacture of a medicament for the treatment of chronic inflammatory diseases such as inflammatory bowel disease, rheumatoid arthritis, synovitis and psoriasis.

As indicated above, component compounds of Aristolochia taliscana have also been found to have good antifungal activity, and in a still further aspect, the invention provides the use of an extract of Aristolochia taliscana or one or more antifungally active compounds isolable therefrom for the manufacture of a composition for antifungal use, for example in the treatment of plants or animals.

The invention also provides pharmaceutical compositions comprising benzofuran compounds of the type found in Aristolochia taliscana or benzofuran compounds analogous thereto, for example benzofuran compounds in which an aryl ring (such as an oxygenated phenyl ring) is attached to the heterocyclic ring of the benzofuran, and the uses of such compounds in medicine.

The invention also provides a novel group of benzofuran compounds having an oxygenated aryl ring (such as an oxygenated phenyl ring) attached to the heterocyclic ring of the benzofuran.

### DESCRIPTION OF PREFERED EMBODIMENTS

Compounds for Use in Medicine—New Medical Uses of Known and Novel Compounds

In one preferred aspect, the invention provides the use of a compound for the manufacture of a medicament for use in any one or more of the therapeutic uses selected from the treatment or alleviation of AIDS or the symptoms thereof, or the alleviation or reversal of cachexia, or the treatment of neoplastic diseases or diseases mediated or intiated by mutagenesis or abnormal cellular proliferation, or as a cytotoxic agent, or the treatment of chronic inflammatory conditions, or the treatment of neurological disorders such as Parkinsonism, or the treatment of male impotence; the compound being of the formula (I):

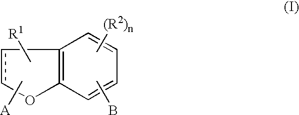

wherein the dotted line signifies a single or double bond; n is 0, 1, 2 or 3; A is a monocyclic aryl ring containing up to two heteroatoms and being optionally substituted by one or more substituent groups which may be the same or different and are selected from R³O, R³, R³S, halogen; aryl and heteroaryl, wherein R³ is hydrogen, or a hydrocarbyl group optionally substituted by a hydroxy or hydrocarbyloxy group; B is selected from carboxy, carboxaldehyde, hydrocarbyl and hydrocarbyloxy groups wherein the hydrocarbyl group is acyclic or cyclic, and optionally contains one or more heteroatoms, and is optionally substituted by one or more hydroxy, alkoxy, alkenyloxy, alkynyloxy, aryloxy, aldehyde, alkanoyl, acetal, hemiacetal and carboxy groups; R¹ is hydrogen or a hydrocarbyl group optionally including one or more heteroatoms and optionally substituted by one or more substituents selected from hydroxy, hydrocarbyloxy and aryl groups; and R² is hydroxy or a hydrocarbyl or hydrocarbyloxy group optionally substituted by one or more substituents selected from hydroxy, hydrocarbyloxy and aryl groups.

It is preferred that the monocyclic aryl ring A is attached to the 2-position of the furan ring, and it is particularly preferred that the aryl ring is a phenyl group. The phenyl ring can contain up to five substituent groups but preferably contains no more than three substituents.

Preferably, the group B is attached to the 5-position of the benzofuran group.

Preferably, there is only one group R², which is attached to the 7-position of the benzofuran ring.

Preferably, the dotted line signifies a double bond.

In a particularly preferred embodiment, the invention provides the use of a compound for the manufacture of a medicament for use in the treatment of the conditions described above in relation to formula (I), the compound having the formula (II):

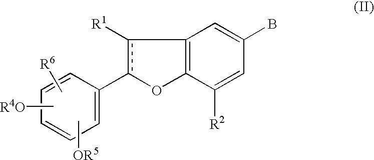

wherein the dotted line signifies a single or double bond, B, R¹ and R² are as hereinbefore defined, R⁴ and R⁵ are the same or different and each is selected from hydrogen, C₁₋₂₀ hydrocarbyl, C₅₋₂₀ aryl, or C₅₋₂₀ oxygen-containing heteroaryl; R⁶ is selected from C₁₋₂₀ hydrocarbyl or C₁₋₂₀ hydrocarbyloxy optionally substituted by one or more hydroxy, alkoxy or aralkyloxy groups; or R⁶ is C₅₋₂₅ aryl or oxygen or nitrogen-containing heteroaryl.

One preferred group of compounds are the compounds in which B is C₁₋₆ alkyl or alkenyl optionally substituted by one or more substituents selected from hydroxy, CHO, or R⁷O wherein R⁷ is a C₁₋₆ alkyl or alkenyl group. More preferably, the group B is selected from CH═CHCH₃, CH₂CH═CH₂, CH(OH)CH═CH₂, CH═CHCHO, CHO, CH═CHCH₂OH and CH(OH)CH(OH)CH₃. A particularly preferred group B is CH═CHCH₃.

In compounds of the formula (II) R⁴ and R⁵ are preferably selected from hydrogen, or C₁₋₆ alkyl, or R⁴ and R₅ together define an alkylene group such as —CH₂—. Preferably, at least one of R⁴ and R⁵ is hydrogen.

Particularly preferred compounds are those in which the dotted line signifies a double bond and one of R⁴ and R⁵ is hydrogen.

Examples of groups R⁶ are hydrogen, halogen, C₁₋₆ alkoxy (e.g.methoxy), a 2-benzofuranyl ring, or an aristolactam group.

In the foregoing formulae (I) and (II), examples of hydrocarbyl groups are aliphatic, alicyclic and aromatic groups such as alkyl, alkenyl, alkynyl, cycloalkyl, cycloalkylalkyl, cycloalkylalkenyl, cycloalkylalkynyl, cycloalkenyl, cycloalkenylalkyl, cycloalkenylalkenyl, aryl, aralkyl, aralkenyl, aralkynyl. The hydrocarbyl groups can be optionally interrupted by one or more heteroatoms such as oxygen and sulphur.

Particular examples of alkyl groups are C₁₋₆ alkyl groups such as methyl, ethyl, propyl, isopropyl, butyl, isobutyl and t-butyl.

Examples of cycloalkyl groups are cyclopropyl, cyclobutyl, cyclopentyl, cyclohexyl, cycloheptyl, cyclooctyl, bicycloheptanyl, decalinyl, adamantyl, norbornyl and bicyclooctyl.

Examples of alkenyl and alkynyl groups include vinyl, ethynyl, allyl, 1-propenyl, propargyl, but-1-enyl, but-2-enyl, but-3-enyl and 3-methylbutenyl.

Examples of cycloalkenyl groups are cyclopentenyl, cyclohexenyl, cycloheptenyl, and monocyclic, bicyclic and tricylic terpene groups.

Examples of aryl groups are phenyl and naphthyl.

Examples of phenylalkyl and phenylalkenyl groups are benzyl, phenethyl, phenylpropyl, phenylbutyl and styryl groups.

First Medical Uses of Compounds Not Previously Disclosed as Having Therapeutic Utility

Many compounds of the formualae (I) and (II) have not previously been disclosed as having any therapeutic uses. Accordingly, in another embodiment, the invention provides a compound of the formula (I) or (II) as hereinbefore defined for use in medicine, for example for use in any one or more of the therapeutic uses selected from the treatment or alleviation of AIDS or the symptoms thereof, or the alleviation or reversal of cachexia, or the treatment of neoplastic diseases or diseases mediated or intiated by mutagenesis or abnormal cellular proliferation, or as a cytotoxic agent, or the treatment of chronic inflammatory conditions, or the treatment of neurological disorders such as Parkinsonism, or the treatment of male impotence, or as an anti-fungal agent in the treatment of plants or animals; but provided that when R¹ is 3-methyl, R² is a single methoxy group at the 7-position, and either (i) the furan ring is unsaturated and is substituted at the 2-position with a 4-hydroxy-3-methoxyphenyl group or a 3,4-methylenedioxyphenyl group; or (ii) the furan ring is a 2,3-dihydrofuran ring and is substituted at the 2-position with a 4-hydroxy-3-methoxyphenyl group, then B is other than a prop-1-enyl group attached to the 5-position of the benzfuran ring.

Novel Compounds per se

The present invention also provides novel compounds per se of the formula (III):

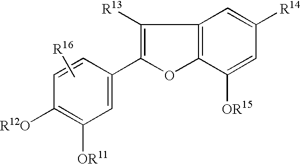

wherein R¹¹ is hydrogen or C₁₋₆ alkyl;

R¹² is selected from hydrogen, C₁₋₆ alkyl; a cyclic terpenoid group or a group of the formula E, G or J;

R¹³ is selected from hydrogen; C₁₋₃ alkyl or hydroxy-C₁₋₃ alkyl;

R¹⁴ is selected from CH═CH—CH₃, CH(OH)CH═CH₂, CH═CH—CHO, CH═CH—CH₂OH, CH(OH)CH(OR¹⁷)CH₃, or a group L;

R¹⁵ is hydrogen or C₁₋₆ alkyl;

R¹⁶ is hydrogen, a group M or an aristolactam group; and

R¹⁷ is hydrogen or a group T; wherein the groups E, G, L, J, M and T are represented by the formulae:

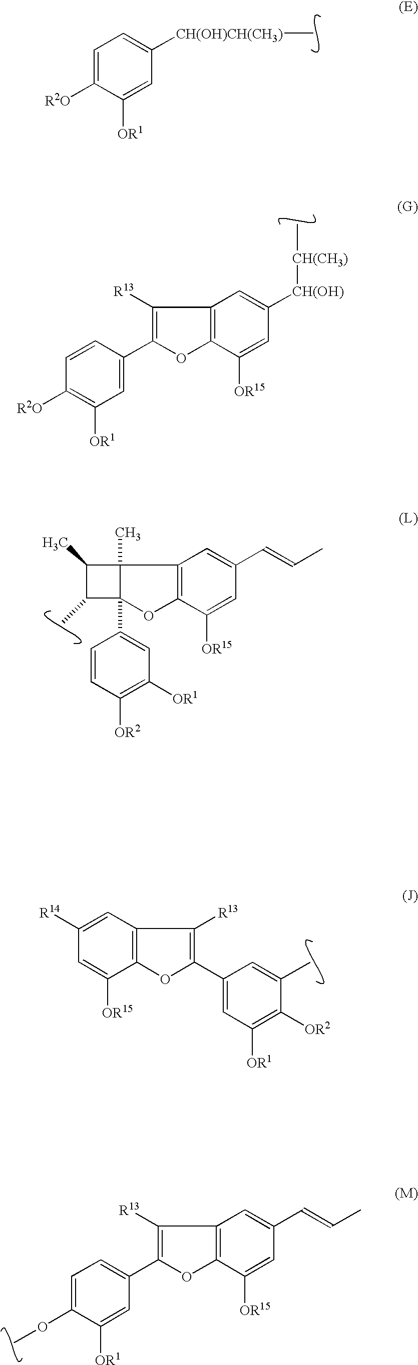

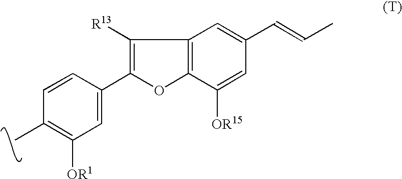

and pharmaceutically acceptable salts thereof; provided that when R¹¹, R¹³ and R¹⁵ are all methyl, and R¹² and R¹⁶ are both hydrogen, R¹⁴ is selected only from CH(OH)CH═CH₂, CH═CH—CHO, CH═CH—CH₂OH, CH(OH)CH(OR¹⁷)CH₃ where R¹⁷ is a group T, or a group L.

In one particular embodiment, there is provided a novel compound of the formula (IV):

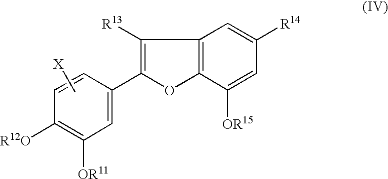

wherein R¹¹, R¹², R¹³ R¹⁴, R¹⁵ and R¹⁷ are as hereinbefore defined and X is a group:

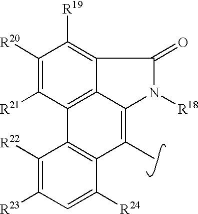

wherein R¹⁸ is hydrogen, benzyl or C₁₋₆ alkyl; R¹⁹ to R²⁴ are the same or different and are selected from hydrogen, hydroxy, C₁₋₆ alkoxy, C₁₋₆ alkyl and hydroxy-C₁₋₆ alkyl; or any two adjacent groups together form an alkylene dioxy group.

In another embodiment, the invention provides novel compounds of the formula (V):

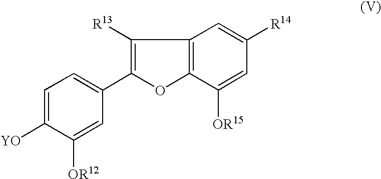

wherein Y is a monocyclic or bicyclic terpenoid group and in particular a group of the structure:

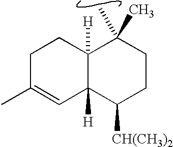

Tetralone Compounds

In a further aspect, the invention provides tetralone compounds for use in medicine, the tetralone compounds being of the formula (VI):

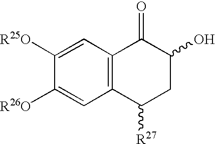

wherei R²⁵ and R²⁷ are the same or different and each is C₁₋₆ alkyl, or R²⁵ and R²⁶ together form an alkylene group (such as methylene); and R²⁶ is hydrogen or C₁₋₆ alkyl.

Preferably R²⁵, R²⁶ and R²⁷ are all methyl.

Tetralone compounds of the formula (VI) have biocidal activity, and in particular cytotoxic, antibacterial and antifungal activity. It is therefore anticipated that they will be useful in the treatment of proliferative and infective diseases and conditions such as cancers and bacterial and fungal infections.

Accordingly, the invention also provides a compound of the formula (VI) for use in the treatment of bacterial or fungal infections, or for use in the treatment of cancers and other proliferative diseases such as psoriasis.

Compounds of the formula (VI) have previously been reported as synthetic intermediates (see loie et al. Chem. Pharm. Bull. 38, 1851-56 (1990).

Particular novel compounds of the invention are:

(±)-5-(1-Hydroxyallyl)-2-(4-hydroxy-3-methoxyphenyl)-7-methoxy-3-methylbenzofuran (Compound 9);

2-(4-Hydroxy-3-methoxyphenyl)-3-hydroxymethyl-7-methoxy-5-(E)-propenylbenzofuran (Compound 10);

2-(4-Hydroxy-3-methoxyphenyl)-7-methoxy-3-methyl-5-[(E)-3-oxopropenyl]benzofuran (Compound 11);

5-Formyl-3-(4-hydroxy-3-methoxyphenyl)-7-methoxy-3-methylbenzofuran (Compound 12);

2-(4-Hydroxy-2-methoxyphenyl)-5-[(E)-3-hydroxypropenyl]-7-methoxy-3-methylbenzofuran (Compound 13);

2-(3,4-Dihydroxyphenyl)-7-methoxy-3-methyl-5-(E)-propenylbenzofuran (Compound 14);

erythro-5-(1,2-Dihydroxypropyl)-2-(4-hydroxy-3-methoxyphenyl)-7-methoxy-3-methylbenzofuran (Compound 15);

(2R,3R)-2,3-Dihydro-2-(4-hydroxy-3-methoxyphenyl)-3-hydroxymethyl-7-methoxy-5-(E)-propenylbenzofuran (Compound 19);

erythro-1-(4-Acetoxy-3-methoxyphenyl)-2-[4-(7-methoxy-3-methyl-5-(E)-propenylbenzofuran-2-yl)-2-methoxyphenoxy]propylacetate (Compound 22);

threo-1-(4-Acetoxy-3-methoxyphenyl)-2-[4-(7-methoxy-3-methyl-5-(E)-propenylbenzofuran-2-yl)-2-methoxyphenoxy]propyl-acetate (Compound 23);

threo-1-[2-(4-Hydroxy-3-methoxyphenyl)-7-methody-3-methylbenzofuran-5-yl]-2-[4-(3-methyl-5-(e)-propenylbenzofuran-2-yl)-2-methoxyphenoxy]propan-1-ol (Compound 24);

2-Methoxy-4-[7-methoxy-3-methyl-5-(E)-propenylbenzofuran-2-yl]-6-[4-(7-methoxy-3-methyl-5-(E)-propenylbenzofuran-2-yl)-2-methoxyphenoxy]phenol (Compound 25)

8.2′,9.3′-Tetrahydro-bis-eupomatenoid-7 (Compound 26);

15-(Aristolactam-I-9-yl)-eupomatenoid-7 (Compound 27);

14-O-α-Cadinyl-eupomatenoid-7 (Compound 28); and

(2R,4S)-2-Hydroxy-6-methoxy-4,7-dimethyl-1-tetralone (Compound 34).

Extraction of Compounds from Aristolochia taliscana

Certain compounds of the formulae I to VI can be obtained by solvent extraction of plant material, such as roots, bark, leaves and twigs, from Aristolochia taliscana using solvents such as benzene followed by chromatographic separation of the components of the solvent extract. A typical extraction protocol is described in detail below.

Synthesis of Compounds of the Formulae I to V

The compounds of the invention, whether naturally occurring or sythetic analogues thereof can be synthesized from readily available starting materials by synthetic methods well known to those skilled in the art.

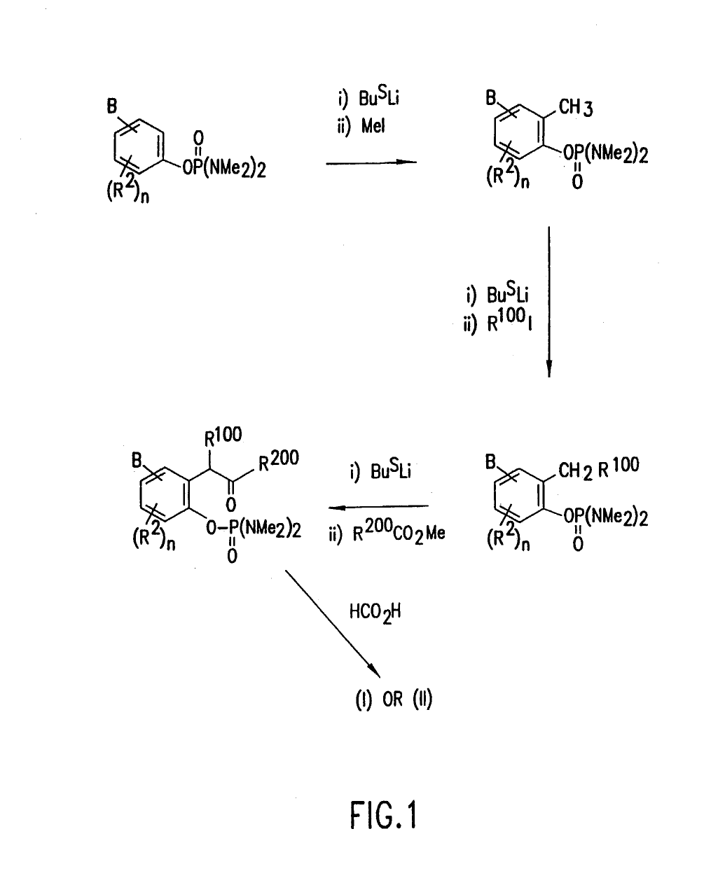

For example, compounds of the formulae (I) or (II) can be prepared by means of the reaction scheme set out in FIG. 1.

The reaction conditions and reagents employed in the scheme set out in FIG. 1 can be substantially as described in M. Watanabe et al. Chem. Pharm. Bull. 37, 2884 (1989); ibid. 38, 41 (1990), and ibid. 39, 3123 (1991), the contents of which are incorporated herein by reference.

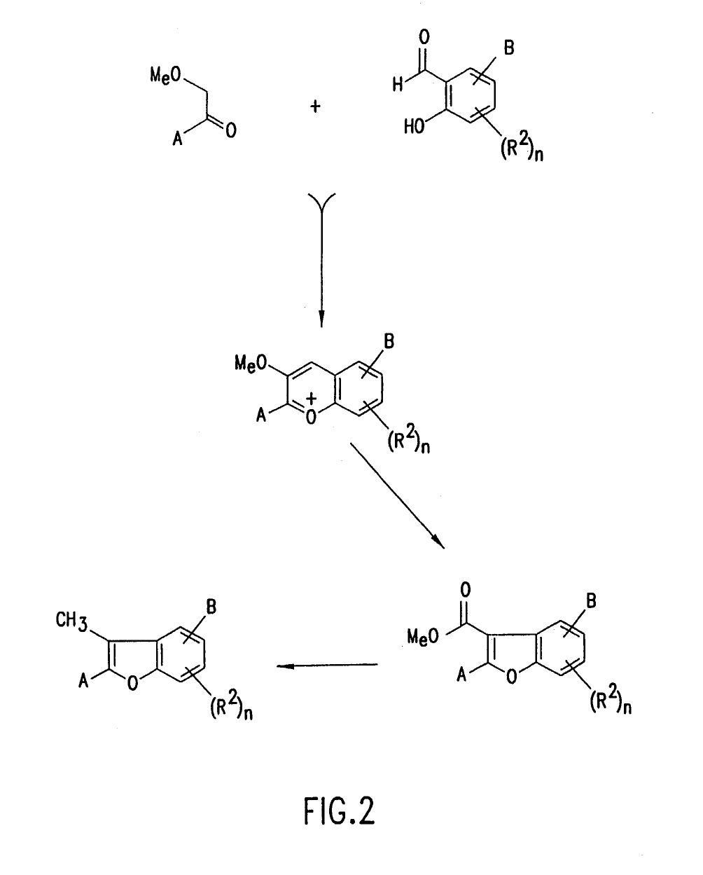

An alternative synthetic scheme applicable to compounds of the formulae (I) or (II) wherein R¹ is a methyl group attached to the 3-position of the furan ring and A is an aryl group attached to the 2-position of the furan ring, is set out in FIG. 2.

In the reaction scheme shown in FIG. 2, the methoxymethylaryl ketone is reacted with the substituted o-hydroxybenzaldehyde in an acidic medium (for example a mixture of hydrochloric acid and acetic acid) to give a benzpyryllium salt which is then subjected to oxidation and rearrangement in the presence of hydrogen peroxide and methanol at pH 5.8 to give a benzfuran 3-carboxy ester. The benzfuran 3-carboxyester can then be treated successively with (i) lithium aluminium hydride in an ether such as diethyl ether; (ii) manganese dioxide in a non-polar solvent such as benzene; (iii) 1,2 ethylene-dithiol, acetic acid and boron trifluoride etherate; and (iv) Raney nickel in an alcohol such as ethanol. The general conditions under which each of the above reactions can be carried out are disclosed in McCredie et al., Austral. J. Chem. 22, 1011 (1969), the contents of which are incorporated herein by reference.

Pharmaceutical Uses

The extracts and compounds of the invention are useful in a number of medical aspects. For example, as indicated above, they are useful in the treatment and management of AIDS. In use as therapeutic agents, for example in the treatment of AIDS, the compounds or extracts can be administered in standard manner, for example orally, parenterally, transdermally, rectally, via inhalation or via buccal administration. Preferably, however, they are administered orally. The dosage employed will depend on the nature and purity of the extract and the concentrations of the active principles. For an extract that has not been fractionated, the concentration administered can be in the range from 0.5 mg to 500 mg (dry weight) of extract per patient per day, more usually 1 mg to 100 mg per day. If an isolated compound or synthetic analogue thereof, or mixture of such compounds is employed, the dosages of such compounds administered typically will be similarly in the range 0.5 mg to 500 mg per patient per day, more usually 1 mg to 100 mg per day. The extracts or compounds may be administered as single doses or multiple doses as desired. The dosages of the extracts or compounds of the invention administered will depend upon inter alia the potency of the extract or compound, and the nature and severity of the disease state or condition under treatment but ultimately, however, will be at the discretion of the physician.

Pharmaceutical Formulations

The extracts and compounds of the invention can be formulated as solutions, syrups, tablets, capsules, lozenges, inserts, patches, powders, pills, solutions for injection or drops, or aerosols such as dry powder aerosols or liquid aerosols, by way of example. Such formulations can be prepared in accordance with methods well known per se.

In a particular embodiment, the compositions of the invention can take the form of solid or semi-solid unit dosage form. For example, the compositions can take the form of tablets, granules, lozenges or capsules.

A solid or semi-solid dosage form according to the present invention can contain, for example, from 10 mg to 1000 mg of the extract or compounds of the invention, more typically 50 mg to 500 mg, e.g. 100 mg to 400 mg, and in particular 150 mg to 350 mg, particular unit dosages being approximately 200 mg and 300 mg.

A tablet composition will typically contain one or more pharmaceutically acceptable solid diluents, examples of which include sugars such as sucrose and lactose, and sugar alcohols such as xylitol, sorbitol and mannitol; lactose and sorbitol being particular examples.

The tablets will also typically contain one or more excipients selected from granulating agents, binders, lubricants and disintegrating agents.

Examples of disintegrants include starch and starch derivatives, and other swellable polymers, for example cross-linked polymeric disintegrants such as cross-linked carboxymethylcellulose, cross-linked polyvinylpyrrolidone and starch glycolates.

Examples of lubricants include stearates such magnesium stearate and stearic acid.

A capsule composition typically will comprise an outer shell or casing which may, for example, be formed from hard or soft forms of gelatin or gelatin-equivalents in conventional fashion. The outer shell is filled with an extract or a compound in accordance with the invention. The capsule filling may be in the form of a powder, or granules, or beads, or may be in the form of a liquid or semi-solid. Where the mixture is in the form of granules, the granules can consist of the extract or compound of the invention alone, or granulated together with a granulating agent, or they can additionally comprise a solid diluent, for example of the type set forth above.

The granules can be wet granulated or dry granulated as desired.

When the capsule filling is in liquid or semi-solid form, the extract or compound can be dissolved or suspended in a semi-solid carrier material such as a polyethylene glycol or a liquid carrier such as a glycol, e.g. propylene glycol, or glycerol. In general, it is preferred that the capsule is in solid or semi-solid form when hard gelatin capsules are used; liquid or semi-solid forms being preferred with soft gelatin capsules.

### DESCRIPTION OF THE PREFERRED EMBODIMENTS

The invention will now be illustrated, but not limited, by reference to the following examples.

### GENERAL EXPERIMENTAL DETAILS AND ISOLATION PROCEDURE

General

In the following examples, all melting points are uncorrected. Analytical thin layer chromatography (TLC) was performed on precoated plates (HPTLC plates, silica gel 50 F₂₅₄, Merck) using the following systems: S-1=CHCl₃—MeOH (99:1), S-2=CHCl₃—MeOH (96:4), S-3=cyclohexane-EtOAc (1:1); detection: UV, anisaldehyde reagent [E. Stahl, and U. Kaltenback, Journal of Chromatography, 1961, 5, 351].

Unless otherwise stated, the optical properties and UV and IR spectra were recorded as follows: [α]_{D} in CHCl₃ at 20°, CD and UV in MeOH, IR in CHCl₃.

Unless otherwise stated, ¹H NMR were run at 360 MHz and ¹³C NMR at 90 MHz in CDCl₃ with TMS as internal standard.

EIMA were obtained at 70 eV; DCIMS with NH₃ or isobutane, respectively. Apart from key ions, the only ions listed are those with relative intensities >10% and m/z>100.

Column chromatography (CC) and medium pressure liquid chromatography (MPLC) were carried out on silica gel 60 (Macherey-Nagel) and on LiChroprep® RP 18 (40-60 μm, Merck). For CC, Fractogel PVA 500 (Merck), and Fractogel TSK HW-40 (S) (Merck) were also used.

High pressure liquid chromatography (HPLC) was performed on LiChrosorb RP 18 (7 μm, Merck).

Plant Material

Roots of Aristolochia taliscana Hook (Aristolochiaceae) were collected by Jorge Pérez de la Rosa (Instituto Tecnologico y de Estudios Superiores de Monterrey, ITESM) from Colima (Mexico) and identified by Prof. H. Sánchez. A voucher specimen is held at the Universidad de Guadalajara, Instituto de Botanica, Guadalajara (Mexico).

### EXAMPLE 1

Extraction and Isolation of the Components of Aristolochia taliscana

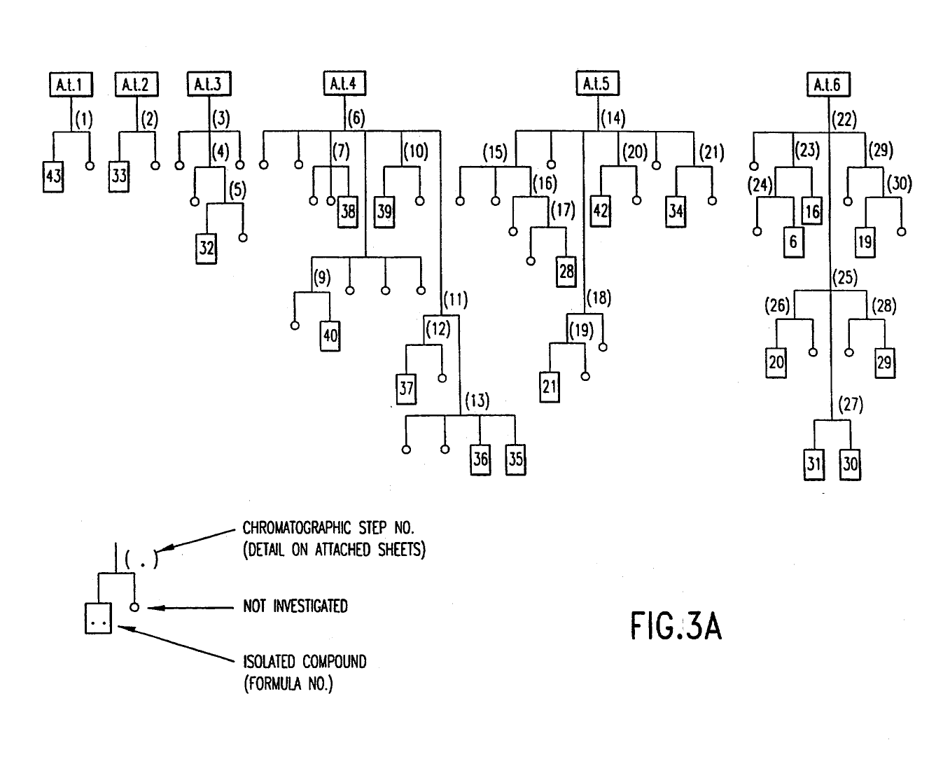

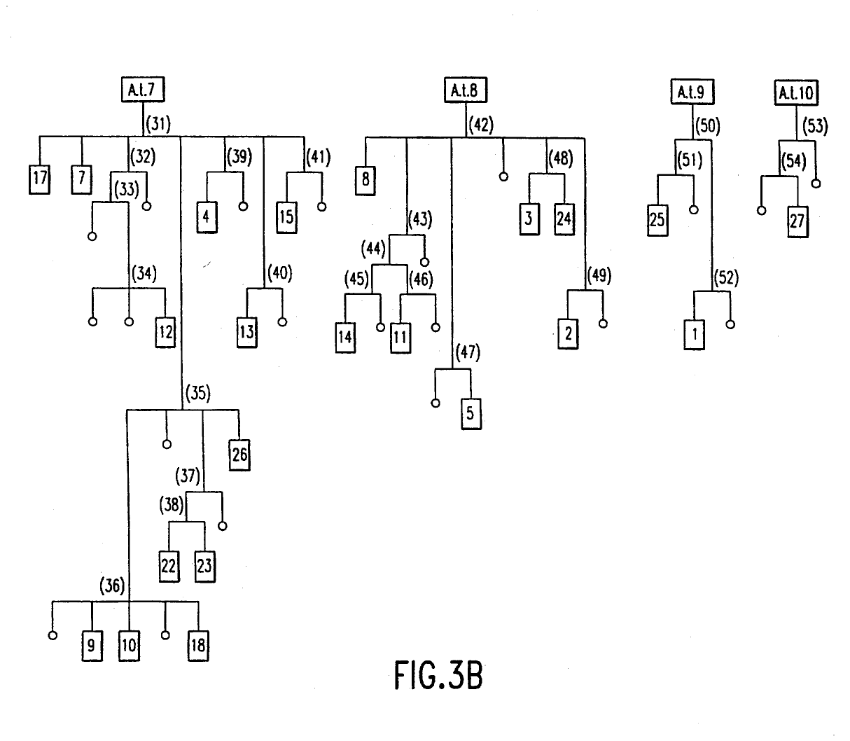

Air dried, pulverized roots and rhizomes (3.5 kg) of Aristolochia taliscana were extracted with benzene at room temperature to give 16 g of a red-brown extract after removal of solvent. This extract was separated by column chromatoraphy on Fractogel TSK HW 40 (S) with methanol to give 10 fractions (designated A.t. 1 to A.t 10), which were then subjected to further chromatographic separation by repeated MPLC or CC using the following systems (a) silica gel, cyclohexane-ethyl acetate gradients, (b) LiChroprep RP 18, MeOH—H₂O gradients, (c) Fractogel PVA 500, methanol. The separation scheme followed is set out in FIG. 3, and the experimental conditions employed in each of the separation steps are set out in Table 2 below.

Purification and final separation was achieved by HPLC on silica gel Nucleosil 50 using cyclohexane-ethyl acetate (8:2) and high pressure liquid chromatography on silica gel RP 18 (LiChrosorb) using methanol-water mixtures, respectively. These procedures afforded the individual compounds 1 to 32 and 34 to 41 besides the mixtures 33, 42 and 43, whose identification was achieved by methylation or methanolysis and subsequent gas chromatographic analysis.

TABLE 2

| Step | Applied to | | | Column | | | |
|---|---|---|---|---|---|---|---|
| No. | Fraction | Adsorbent | Eluent | dimensions | | Fractions obtained | |
| 1 | A.t.1 | Silica gel | Gradient | D | 1.2 cm | 1 | 246 mg = 43 |
| | 361 mg | 40 g | CH/EA | L | 46 cm | 2 | 63 mg ∘ |
| | | | 10/0 | | | 3 | 39 mg ∘ |
| | | | 0/10 | | | | |
| 2 | A.t.2 | Silica gel | Gradient | D | 1.2 cm | 1 | 180 mg = 33 |
| | 431 mg | 40 g | CH/EA | L | 46 cm | 2 | 232 mg ∘ |
| | | | 10/0 | | | 3 | 15 mg ∘ |
| | | | 0/10 | | | | |
| 3 | A.t.3 | Silica gel | Gradient | D | 2.5 cm | 1 | 209 mg ∘ |
| | 904 mg | 160 g | | L | 46 cm | 2 | 611 mg = A.t.3.2 |
| 4 | A.t.3.2 | Silica gel | CH/EA | D | 2.5 cm | 1 | 150 mg ∘ |
| | 611 mg | 160 g | 7/3 | L | 46 cm | 2 | 431 mg = A.t. 3.2.2 |
| 5 | A.t. 3.2.2 | Nucleosil | M/EtOH | D | 2 cm | 1 | 55 mg = 32 |
| | 60 mg | RP-18, 7 μm | 9/1 | L | 25 cm | 2 | 4 mg ∘ |
| 6 | A.t.4 | Silica gel | Gradient | D | 5 cm | 1 | 39 mg ∘ |
| | 1079 mg | 640 mg | CH/EA | L | 46 cm | 2 | 10 mg ∘ |
| | | | 10/0 | | | 3 | 156 mg = A.t. 4.3 |
| | | | | | | 4 | 308 mg = A.t. 4.4 |
| | | | 0/10 | | | 5 | 322 mg = A.t. 4.5 |
| | | | | | | 6 | 51 mg = A.t. 4.6 |
| 7 | A.T. 43 | Nucleosil | M/W | D | 2 cm | 1 | 45 mg ∘ |
| | 156 mg | RP-18, 7 μm | 96/4 | L | 25 cm | 2 | 63 mg ∘ |
| | | | | | | 3 | 28 mg = 38 |
| 8 | A.t. 4.4 | LiChroprep | Gradient | D | 1.2 cm | 1 | 235 mg = A.t. 4.4.1 |
| | 308 mg | RP-18, 40 g | M/W | L | 46 cm | 2 | 31 mg ∘ |
| | | | 8/2 | | | 3 | 9 mg ∘ |
| | | | | | | 4 | 17 mg ∘ |
| | | | 10/0 | | | | |
| 9 | A.t. 4.4.1 | Nucleosil | M/W | D | 2 cm | 1 | 90 mg ∘ |
| | 120 mg | RP-18, 7 μm | 84/16 | L | 25 cm | 2 | 28 mg = 40 |
| 10 | A.t. 4.5 | Silica Gel Si | H/iso-PrOH | D | 2 cm | 1 | 15 mg ∘ |
| | 30 mg | 60, | 98/2 | L | 25 cm | 2 | 11 mg = 39 |
| | | 10 μm | | | | | |
| 11 | A.t. 4.6 | LiChroprep | M/W | D | 1.2 cm | 1 | 23 mg = A.t. 4.6.1 |
| | 51 mg | RP-18, 40 g | 8/2 | L | 46 cm | 2 | 20 mg ∘ |
| 12 | A.t. 4.6.1 | Nucleosil | M/W | D | 2 cm | 1 | 9 mg = 37 |
| | 23 mg | RP-18, 7 μm | 9/1 | L | 25 cm | 2 | 5 mg ∘ |
| 13 | A.t. 4.6.2 | Nucleosil | M/W | D | 2 cm | 1 | 1 mg ∘ |
| | 20 mg | RP-18, 7 μm | 98/2 | L | 25 cm | 2 | 1 mg ∘ |
| | | | | | | 3 | 13 mg = 36 |
| | | | | | | 4 | 4 mg = 35 |
| 14 | A.T. 5 | Silica gel | Gradient | D | 2.5 cm | 1 | 115 mg = A.t. 5.1 |
| | 784 mg | 160 g | CH/EA | L | 46 cm | 2 | 121 mg ∘ |
| | | | 8/2 | | | 3 | 60 mg = A.t. 5.3 |
| | | | | | | 4 | 201 mg = A.t. 5.4 |
| | | | 0/10 | | | 5 | 145 mg ∘ |
| | | | | | | 6 | 54 mg = A.t. 5.6 |
| 15 | A.t. 5.1 | PVA-500 | MeOH | D | 1 cm | 1 | 72 mg ∘ |
| | 115 mg | 30 g | | L | 100 cm | 2 | 8 mg ∘ |
| | | | | | | 3 | 38 mg = A.t. 5.1.3 |
| 16 | A.t. 5.1.3 | PVA-500 | MeOH | D | 1 cm | 1 | 5 mg ∘ |
| | 38 mg | 15 g | | L | 46 cm | 2 | 30 mg = A.t 5.1.3.2 |
| 17 | A.t. 5.1.3.2 | Nucleosil | M/W | D | 0.8 cm | 1 | 21 mg ∘ |
| | 30 mg | RP-18, 7 μm | 95/5 | L | 25 cm | 2 | 4 mg = 28 |
| 18 | A.t. 5.3 | FVA-500 | MeOH | D | 1 cm | 1 | 20 mg = A.t. 5.3.1 |
| | 60 mg | 15 g | | L | 46 cm | 2 | 35 mg ∘ |
| 19 | A.t. 5.3.1 | PVA-500 | MeOH | D | 1 cm | 1 | 17 mg = 21 |
| | 20 mg | 15 g | | L | 46 cm | 2 | 1 mg ∘ |
| 20 | A.t. 5.4 | PVA-500 | MeOH | D | 2.5 cm | 1 | 53 mg = 42 |
| | 201 mg | 100 g | | L | 100 cm | 2 | 120 mg ∘ |
| 21 | A.t. 5.6 | LiChroprep | M/W | D | 1.2 cm | 1 | 18 mg = 34 |
| | 54 mg | RP-18, 40 g | 1/1 | L | 46 cm | 2 | 31 mg ∘ |
| 22 | A.t. 6 | Silica gel | Gradient | D | 2.5 cm | 1 | 3 mg ∘ |
| | 1750 mg | 160 g | CH/EA | L | 46 cm | 2 | 1549 mg = A.t. 6.2 |
| | | | 8/2 | | | 3 | 79 mg = A.t. 6.3 |
| | | | | | | 4 | 115 mg = A.t. 6.4 |
| | | | 5/5 | | | | |
| 23 | A.t. 6.2 | LiChroprep | M/W | D | 2.5 cm | 1 | 3 mg = A.t. 6.2.1 |
| | 1549 mg | RP-18, 160 | 7/3 | L | 46 cm | 2 | 1540 mg = 16 |
| | | g | | | | | |
| 24 | A.t. 6.2.1 | Nucleosil | M/W | D | 2 cm | 1 | <1 mg ∘ |
| | 3 mg | RP-18, 7μm | 75/25 | L | 25 cm | 2 | 2 mg = 6 |
| 25 | A.t. 6.3 | Silica gel | CHCl₃ | D | 1 cm | 1 | 30 mg = A.t. 6.3.1 |
| | 79 mg | 9 g | | L | 20 cm | 2 | 29 mg = A.t. 6.3.2 |
| | | | | | | 3 | 11 mg = A.t. 6.3.3 |
| 26 | A.t. 6.3.1 | LiChroprep | M/W | D | 1.2 cm | 1 | 4 mg = 20 |
| | 30 mg | RP-18, 40 g | 6/4 | L | 46 cm | 2 | 21 mg ∘ |
| 27 | A.t. 6.3.2 | LiChroprep | M/W | D | 1.2 cm | 1 | 27 mg = 31 |
| | 29 mg | RP-18, 40 g | 55/45 | L | 46 cm | 2 | 2 mg = 30 |
| 28 | A.t. 6.3.3 | PVA 500 | MeOH | D | 1 cm | 1 | <1 mg ∘ |
| | 11 mg | 15 g | | L | 40 cm | 2 | 10 mg = 29 |
| 29 | A.t. 6.4 | Silica gel | CHCl₃ | D | 1.2 cm | 1 | 11 mg ∘ |
| | 115 mg | 40 g | | L | 46 cm | 2 | 16 mg = A.t. 6.4.2 |
| | | | | | | 3 | 73 mg = A.t. 6.4.3 |
| | | | | | | 4 | 5 mg = A.t. 6.4.4 |
| 30 | A.t. 6.4.2 | Nucleosil | M/W | D | 2 cm | 1 | 7 mg = 19 |
| | 16 mg | 40 g | 6/4 | L | 25 cm | 2 | 6 mg ∘ |
| 31 | A.t. 7 | Silica gel | CH/EA | D | 5 cm | 1 | 1290 mg = 17 |
| | 6177 mg | 640 g | 6/4 | L | 46 cm | 2 | 4350 mg = 7 |
| | | | | | | 3 | 40 mg = A.t. 7.3 |
| | | | 3/7 | | | 4 | 91 mg = A.t. 7.4. |
| | | | | | | 5 | 52 mg = A.t. 7.5 |
| | | | | | | 6 | 11 mg = A.t. 7.6 |
| | | | | | | 7 | 328 mg = A.t. 7.7 |
| 32 | A.t. 7.3 | LiChroprep | M/W | D | 1.2 cm | 1 | 24 mg = A.t. 7.3.1 |
| | 40 mg | RP-18, 40 g | 5/5 | L | 46 cm | 2 | 7 mg ∘ |
| | | | 9/1 | | | | |
| 33 | A.t. 7.3.1 | LiChroprep | M/W | D | 1.2 cm | 1 | 13 mg ∘ |
| | 24 mg | RP-18, 40 g | 3/7 | L | 46 cm | 2 | 10 mg = A.t. 7.3.1.2 |
| 34 | A.t. 7.3.1.2 | Nucleosil | M/W | D | 2 cm | 1 | 2 mg ∘ |
| | 10 mg | RP-18, 7 μm | 75.25 | L | 25 cm | 2 | 3 mg ∘ |
| | | | | | | 3 | 2 mg = 12 |
| 35 | A.t. 7.4 | LiChroprep | Gradient | D | 1.2 cm | 1 | 42 mg =A.T. 7.4.1 |
| | 91 mg | RP-18, 40 g | M/W | L | 46 cm | 2 | 5 mg ∘ |
| | | | 5/5 | | | 3 | 17 mg = A.t. 7.4.3 |
| | | | 9/1 | | | 4 | 4 mg = 26 |
| 36 | A.t. 7.4.1 | Nucleosil | M/W | D | 2 cm | 1 | 3 mg ∘ |
| | 42 mg | RP-18, 7 μm | 7/3 | L | 25 cm | 2 | 7 mg = 9 |
| | | | | | | 3 | 13 mg = 10 |
| | | | | | | 4 | <1 mg ∘ |
| | | | | | | 5 | 2 mg = 18 |
| 37 | A.t. 7.4.3 | TSK HW | MeOH | D | 1 cm | 1 | 11 mg = A.t. 7.4.3.1 |
| | 13 mg | 50s | | L | 100 cm | 2 | 1 mg ∘ |
| | | ca. 100 ml | | | | | |
| 38 | A.t. 7.4.3.1 | LiChrosorb | CH/EA | D | 2 cm | 1 | 6 mg = 22 |
| | 11 mg | Si 60, | 8/2 | L | 25 cm | 2 | 3 mg = 23 |
| | (acetyliert) | 10 μm | | | | | |
| 39 | A.t. 7.5 | LiChroprep | Gradient | D | 1.2 cm | 1 | 30 mg = 4 |
| | 52 mg | RP-18, 40 g | M/W | L | 46 cm | 2 | 9 mg ∘ |
| | | | 5/5 | | | | |
| | | | 9/1 | | | | |
| 40 | A.t. 7.6 | LiChroprep | M/W | D | 1.2 cm | 1 | 4 mg = 13 |
| | 11 mg | RP-18, 40 g | 5/5 | L | 46 cm | 2 | 6 mg ∘ |
| 41 | A.t. 7.7 | LiChroprep | Gradient | D | 1.2 cm | 1 | 4 mg = 15 |
| | 328 mg | RP-18, 40 | M/W | L | 46 cm | 2 | 189 mg ∘ |
| | | mg | 1/1 | | | 3 | 103 mg ∘ |
| | | | 10/0 | | | | |
| 42 | A.t. 8 | Silica gel | Gardient | D | 1.2 cm | 1 | 384 mg = 8 |
| | 771 mg | 40 g | CH/EA | L | 46 cm | 2 | 165 mg = A.t. 8.2 |
| | | | 8/2 | | | 3 | 44 mg = A.t. 8.3 |
| | | | 5/5 | | | 4 | 93 mg = A.t. 8.4 |
| | | | 0/10 | | | 5 | 34 mg = A.t. 8.5 |
| | | | | | | 6 | 8 mg = A.t.8.6 |
| 43 | A.t. 8.2 | LiChroprep | M/W | D | 1.2 cm | 1 | 80 mg = A.t. 8.2.1 |
| | 165 mg | RP-18, 40 g | 75/25 | L | 46 cm | 2 | 74 mg ∘ |
| 44 | A.t. 8.2.1 | Silica gel | C/M | D | 1.2 cm | 1 | 15 mg = A.T. 8.2.1.1 |
| | 80 mg | 40 g | 99/1 | L | 46 cm | 2 | 20 mg = A.t. 8.2.1.2 |
| | | | | | | 3 | 36 mg ∘ |
| 45 | A.t. 8.2.1.1 | PVA 500 | M/C | D | 1 cm | 1 | 9 mg = 14 |
| | 15 mg | 15 g | 9/1 | L | 45 cm | 2 | 4 mg ∘ |
| 46 | A.t. 8.2.1.2 | Pr\overline{a}parA.t.ive | C/M | Laufstrecke | | 1 | 8 mg = 11 |
| | 20 mg | Silica gel- | 99.5/0.5 | 10 cm | | 2 | 11 mg ∘ |
| | | DC | | | | | |
| 47 | A.t. 8.3 | Nucleosil | M/W | D | 2 cm | 1 | 7 mg = A.t. 8.3.1 |
| | 44 mg | RP-18, 7 μm | 83/17 | L | 25 cm | 2 | 8 mg = 5 |
| | | | | | | 3 | 19 mg ∘ |
| 48 | A.t. 8.5 | Nucleosil | M/W | D | 2 cm | 1 | 28 mg = 3 |
| | 34 mg | RP-18, 7 μm | 9/1 | L | 25 cm | 2 | 3 mg = 24 |
| 49 | A.t. 8.6 | PVA 500 | MeOH | D | 1 cm | 1 | 3 mg − 2 |
| | 8 mg | 15 g | | L | 45 cm | 2 | 4 mg ∘ |
| 50 | A.t. 9 | Silica gel | Gradient | D | 2.5 cm | 1 | 56 mg = A.t. 9.1 |
| | 229 mg | 80 g | CH/EA | L | 23 cm | 2 | 20 mg - A.t. 92. |
| | | | 8/2 | | | 3 | 136 mg ∘ |
| | | | 5/5 | | | | |
| | | | 0/10 | | | | |
| 51 | A.t. 9.1 | Nucleosil | M/W | D | 2 cm | 1 | 9 mg = 25 |
| | 56 mg | RP-18, 7 μm | 96.4 | L | 25 cm | 2 | 33 mg ∘ |
| 52 | A.t. 9.2 | Nucleosil | M/W | D | 2 cn | 1 | 4 mg = 1 |
| | 20 mg | RP-18, 7 μm | 9/1 | L | 25 cm | 2 | 13 mg ∘ |
| 53 | A.t. 10 | Silica gel | C/M | D | 1.2 cm | 1 | 23 mg = A.t. 10.1 |
| | 266 mg | 40 g | 10/0 | L | 46 cm | 2 | 141 mg ∘ |
| | | | 95/5 | | | 3 | 83 mg ∘ |
| 54 | A.t. 10.1 | Silcia gel | T/EA | D | 1 cm | 1 | 18 mg ∘ |
| | 23 mg | 9 g | 6/4 | L | 18 cm | 2 | 4 mg = 27 |

Abbreviations:
D: diameter
L: length
CH: cyclohexane
EA: ethyl acetate
H: hexane
M: methanol
T: toluene
W: water

The compounds isolated from the benzene extract are listed below in Table 3. Those compounds already known as natural products are referred to in Table 3 by their chemical names, whilst those compounds not previously recognised as natural products are identified by code number. The full chemical names and spectroscopic and other characterising data for the new natural products are given in the paragraphs following Table 3.

TABLE 3
Compounds isolated from the Benzene Extract of the
Root of Aristolochia taliscana

| Compound Type | Compound (Compound No.) | Content (%)* |
|---|---|---|
| Alkaloid | Aristolactam I (1)* | 0.03 |
| | Aristolactam A III (2) | 0.02 |
| | Aristolactam B III (3) | 0.2 |
| | Aristolactam C III (4) | 0.2 |
| | Taliscanine (5) | 0.06 |
| Lignans | Machilin-F (6) | 0.02 |
| Neolignans | | |
| Benzofuran-type | Eupomatenoid-7 (7) | 34 |
| | Eupomatenoid-1 (8) | 3 |
| | Compound 9 | 0.05 |
| | Compound 10 | 0.1 |
| | Compound 11 | 0.06 |
| | Compound 12 | 0.02 |
| | Compound 13 | 0.03 |
| | Compound 14 | 0.07 |
| | Compound 15 | 0.03 |
| Dihydro-benzofuran type | ( )-Licarin A (16) | 12 |
| | (−)(2S,3S)-Eupomatenold-8 | 10 |
| | (17) | |
| | (−)(2S,3S)-Machilin-B (18) | 0.02 |
| | Compound 19 | 0.05 |
| | (−)(2S,3S)-5- | 0.03 |
| | Methoxylicarin-A (20) | |
| | (+)(2R,3R)- | 0.1 |
| | Dihydrocarinatidin (21) | |
| Oligomers | Compound 22 | 0.05 |
| | Compound 23 | 0.02 |
| | Compound 24 | 0.02 |
| | Compound 25 | 0.07 |
| | Compound 26 | 0.03 |
| Hybrids | Compound 27 | 0.03 |
| | Compound 28 | 0.03 |
| Phenylpropanes | Coniferyl alcohol (29) | 0.08 |
| | Ferulaaldehyde (30) | 0.02 |
| | Vanillin (31) | 0.2 |
| Sterols | Beta-sitosterol (32) | 0.4 |
| | Mixture of 3-O-acyl-beta- | 1.4 |
| | sitosterols (33) | |
| Terpenoids | Compound 34 | 0.1 |
| | Sandaracopimaradiene (35) | 0.03 |
| | Beta-caryophyllene (36) | 0.1 |
| | Caryopyhilene oxide (37) | 0.07 |
| | ent-Germacrene-D (38) | 0.2 |
| | ent-Germacra-4(15), 5, 10 | 0.09 |
| | (14)-trien-1-beta- | |
| | ol (39 | |
| | Spathulenol (40) | 0.2 |
| Others | D-fructose (41) | 1.3 |
| | Mixture of fatty acids (42) | 0.4 |
| | Mixture of triglcerides (43) | 1.9 |

No aristolochic acids were detected in the extract. The aristolactams referred to in the table have the following structural formulae:

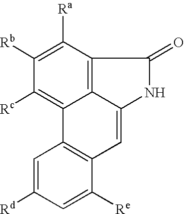

| | R^{a} | R^{b} | R^{c} | R^{d} | R^{e} |
|---|---|---|---|---|---|
| Aristolactam I | H | O—CH₂—O | | H | OCH₃ |
| Aristolactam A III | H | OH | OCH₃ | OCH₃ | H |
| Aristolactam B III | H | OCH₃ | OCH₃ | OCH₃ | H |
| Aristolactam C III | CH₂OH | OCH₃ | OCH₃ | OCH₃ | H |
| Taliscanine | H | OCH₃ | OCH₃ | OCH₃ | H |

### Physico-chemical and Spectroscopic Properties of the Novel Natural Products

### (±)-5-(1-Hydroxyallyl)-2-(4-hydroxy-3-methoxyphenyl)-7-methoxy-3-methylbenzofuran (Compound 9).

Crystals (5 mg). Mp 164-167° (from MeOH). TLC: R_{f} 0.42(S-1); anisaldehyde: violet. [α]_{D}±0° (c.0.1). IRνₘₐₓcm⁻¹:3540(OH), 3020, 1515. UVλₘₐₓ nm(logε):221(3.42), 305(3.38); +NaOH:212(3.82), 328(3,46). ¹H NMR(250 MHz): δ2.00(1H, d, J=3.5 Hz, OH-8), 2.41(3H,s,Me-3), 3.99(3H,s,OMe), 4.03(3H,s,OMe), 5.23(1H,dt,J₁=10.5,J₂=1.5 Hz,H-10_{B}), 5.31(1H,m,H-8), 5.41(1H,dt,J₁=17,J₂=1.5 Hz,H-10_{A}), 5.75 (1H,s,OH-14), 6.14(1H,ddd,J₁=17,J₂=10.5,J₃=6 Hz,H-9), 6.83 (1H,d,J=1.5 Hz,H-6), 7.00(1H,d,J=8 Hz,H-15), 7.12(1H,d,J=1.5 Hz, H-4, 7.29 (1H,dd,J₁=8,J₂=2 Hz,H-16), 7.33(1H,d,J=2 Hz,H-12). ¹³C NMR (60 MHz): δ9.6(Me-3), 56.5(2×OMe), 76.5(C-8), 106.6 (C-6), 110.0(C-12), 110.9(C-3), 111.3(C-4), 114.6(C-15), 116.5(C-10), 121.2(C-16), 124.5(C11), 134.1(C3a), 140.1(C-5), 142.6(C-9), 143.3(C-7a), 146.2(C-7), 148.1(C-14), 149.2(C-13), 152.9(C-2). EIMS m/z (rel. int.): 340[M]⁺(100), 323 (14), 297 (11), 295(11), 284(12).

### 2-(4-Hydroxy-3-methoxyphenyl)-3-hydroxymethyl-7-methoxy-5-(E)-propenylbenzofuran (Compound 10)

Crystals (12 mg). Mp 175-179° (from MeOH). TLC:R_{f}0.3(S-1); anisaldehyde:grey. IRνₘₐₓ cm⁻¹:3539(OH), 1600, 1515, 1466. UVλₘₐₓ nm(logε):231 (3.38), 266(3.44), 304(3.34); +NaOH:240 (3.41), 295(3.25), 328(3.3). ¹H NMR (250 MHz): δ1.57(1H,t,J=4 Hz, OH-3), 1.90(3H,dd,J₁=6.5J₂=1.5 Hz, Me-10), 3.95(3H,s,OMe), 4.04(3H,s,OMe), 4.91(2 H, d, J=4 Hz, CH₂OH), 5.81 (1 H, s, OH-14), 6.23(1H,dq,J₁=16,J₂=6.5 Hz,H-9), 6.48(1H,dq,J₁=16,J₂1.5 Hz,H-8), 6.83(1H,d,J=1.5 Hz,H-6), 7.01(1H,d,J=8 Hz,H-15), 7.18(1H,d,J=1.5 Hz,H-4), 7.38(1H,dd,J₁=8,J₂=2 Hz,H-16), 7.41(1H,d,J=2 Hz,H-12). ¹³C NMR: δ18.4(Me-10), 55.7(CH₂OH), 56.1(2×OMe), 104.8(C-6), 109.0(C-4), 110.0(C-12), 113.8(C-3), 114.7(C-15), 121.3(C-16), 122.4(C-11), 124.8(C-9), 131.2(C-3a), 131.3(C-8), 123.3(C-5), 142.3(C-7a), 145.0(C-7), 146.6(C-14), 146.7(C-13), 154.6(C-2). EIMS m/z(rel.int):340[M]⁺(100), 323(15), 291(19), 151(10).

2-(4-Hydroxy-3-methoxyphenyl)-7-methoxy-3-methyl-5-[(E)-3-oxopropenyl]benzofuran (Compound 11)

Crystals (8 mg). Mp 169-170° (from MeOH). TLC:R_{f}0.39(S-1); anisaldehyde:blue. IR νₘₐₓ cm⁻¹:3538(OH), 1672(CO), 1610, 1514. UV λₘₐₓ nm(logε):213(4.04), 291(4.91), 314(4.31); +NaOH:215(4.93), 337(4.36). ¹H NMR(250 MHz): δ2.46(3H,s,Me-3), 3.99(3H,s,OMe), 4.08(3H,s,OMe), 6.73(1H,dd,J₁=16,J₂=8 Hz,H-8), 7.00(1H,d,J=2 Hz,H-6), 7.04(1 H, d, J=2 Hz, H-15), 7.30(1 H, d, J=8 Hz, H-16), 7.32(1H,dd,J₁=8,J₂=2Hz,H-12), 7.33(1H,d,J=2 Hz,H-4), 7.58(1H,d,J=16 Hz,H-8), 9.72(1H,d,J=8 Hz,CHO). ¹³C NMR(60 MHz): δ9.5(Me-3), 56.1(2×OMe), 105.7(C-6), 109.6(C-12), 110.1(C-3), 113.8(C-4), 114.7(C-15), 120.8(C-16), 122.9(C-11), 124.5(C-9), 129.7(C-5), 133.5(C-3a), 144.7(C-7a), 145.4(C-7), 146.3(C-14), 146.8(C-13), 152.6(C-2), 153.9(C-8), 193.6(C-10). EIMS m/z (rel. int.): 338[M]⁺(96), 311(19), 310(100), 295(28), 267(29), 178(10), 169(12), 165(12), 152(11).

### 5-Formyl-3-(4-hydroxy-3-methoxyphenyl)-7-methoxy-3-methylbenzofuran (Compound 12)

Crystals (2 mg). Mp 162-165° (from MeOH). TLC:R_{f}0.43(S-1); anisaldehyde:light blue. IRνₘₐₓ cm⁻¹:3540(OH), 3023, 1688(CO), 1515. UV λₘₐₓ nm(logε):231(4.40), 283(4.61), 307(sh,4.53); +NaOH:240(4.44), 329(4.62). ¹H NMR: δ2.48(3H,s,Me-3), 4.00(3H,s,OMe), 4.07(3H,s,OMe), 5.80(1H,s,OH), 7.03(1H,d,J=8 Hz,H-15), 7.32(1H,dd,J₁=8, J₂=2 Hz,H-16), 7.33(1H,d,J=2 Hz,H-12), 7.37(1H,d,J=1.5Hz,H-4), 7.68(1H,d,J=1.5 Hz,H-6), 10.0(1H,s,CHO). ¹³C NMR: δ9.5(Me-3), 56.1(2×OMe), 104.7(C-6), 110.5(C-3), 114.6(C-4), 117.4(C-15), 120.8(C-16), 122.8(C-11), 132.9(C-3a), 133.1(C-5), 145.8(C-7), 146.2(C-14), 146.4(C-13), 146.8(C-7a), 153.0(C-2), 192.0(CO). EIMS m/z (rel. int.):312[M]⁺(100), 297(14), 269(12), 156(15).

### 2-(4-Hydroxy-3-methoxyphenyl)-5-[(E)-3-hydroxypropenyl]-7-methoxy-3-methylbenzofuran (Compound 13)

Crystals (7 mg). Mp 180-183° (from MeOH). TLC:R_{f}0.16(S-1); anisaldehyde:violet. IRνₘₐₓ cm⁻¹:3540(OH), 3020, 1612, 1515. UV λₘₐₓ nm(logε):232(3.16), 271(3.30), 306(sh3.23); +NaOH:240(3.29), 291(3,19), 329(3.33). ¹H NMR δ1.45(1H,t,J=5.5 Hz, OH-10), 2.41(3H,s,Me-3), 3.99(3H,s,OMe), 4.05(3H,s,OMe), 4.35(2H,dd,J₁=5.5,J₂=1 Hz,CH₂OH), 5.75(1H,s,OH-14), 6.37)1H dt,J₁=16,J₂=5.5 Hz,H-9), 6.71(1H,dt,J₁=16,J₂=1 Hz,H-8), 6.88(1H,d,J=1.5 Hz,H-6), 7.00(1H,d,J=8.5 Hz,H-15), 7.11 (1 H,d,J=1.5 Hz,H-4), 7.29(1H,dd,J₁=8.5,J₂=2 Hz,H-16), 7.32(1H,d,J=2 Hz, H-12). ¹³C NMR: δ9.6(Me-3), 56.1(2×OMe), 63.8(C-10), 104.8(C-6), 109.5(C-4), 110.2(C-3 and C-12), 114.4(C-15), 120.7(C-16), 123.5(C-11), 127.2(C-9), 132.1(C-8), 132.3(C-3a), 133.2(C-5), 142.6(C-7a), 145.0(C-7), 145.8(C-14), 146.6(C-13), 151.8(C-2). EIMS m/z (rel. int.): 340[M]⁺(100), 312(12), 311(20), 297(22), 284(37), 282(15), 281(12), 279(11), 165(13), 151(14), 149(10), 55(10).

### 2-(3,4-Dihydroxyphenyl)-7-methoxy-3-methyl-5-(E)-propenylbenzofuran (Compound 14)

Oil (9 mg). TLC:R_{f}0.15(S-2); anisaldehyde:grey. IR νₘₐₓ cm⁻¹:3548(OH), 3015, 1600, 1523, 1483. UV λₘₐₓnm(logε):231(4.49), 264(4.59), 207(sh.4.46); +NaOH:242(4.60), 327(4.44). ¹HNMR(CD₃OD, 250 MHz): δ1.88(3H,dd,J₁=6.5,J₂=1.5 Hz,Me-10), 2.37(3H,s,Me-3), 4.01(3H,s,OMe), 6.22(1H,dq,J₁=16,J₂=6.5 Hz,H-9), 6.47(1H,dq,J₁=16,J₂=1.5 Hz,H-8), 6.85(1H,d,J=1.5 Hz,H-6), 6.88(1H,d,J=8.5 Hz,H-15), 7.01(1H,d,J=1.5 Hz,H-4), 7.14(1H,dd,J₁=8.5 Hz,J₁=2Hz,H-16), 7.26(1H,d,J=2 Hz,H-12). ¹³C NMR(CD₃OD,60 MHz): δ9.6(Me-3), 18.6(Me-10), 56.7(OMe), 105.8(C-6), 110.1(C-4), 110.5(C-3), 114.9(C-12), 116.6(C-15), 119.9(C-16), 124.5(C-11), 124.8(C-9), 132.9(C-8), 134.4(C-3a), 135.1 (C-5), 143.3(C-71), 146.2(C-7), 146.5(C-14), 146.9(C-13), 152.9(C-2). EIMS m/z (rel. int.):310[M]⁺(100), 309(10).

### erythro-5-(1,2-Dihydroxypropyl)-2-(4-hydroxy-3-methoxyphenyl)-7-methoxy-3-methylbenzofuran (Compound 15)

Amorphous (3 mg). TLC:R_{f}0.16(S-2); anisaldehyde:violet. [α)_{D}+17° (c0.2). IR νₘₐₓ cm⁻¹:3435(OH), 2927, 1655, 1516, 1462. UV λₘₐₓnm(logε):216(4.16), 304(4.04): +NaOH:211 (4.80), 328(4.11). ¹H NMR: δ1.11(3H,d,J=6.5 Hz,Me-10), 2.42(3H,s,Me-3), 2.44(1H,br d,J=3 Hz, OH-9), 2.61(1H,br d,J=3 HZ,OH-8), 3.90(1H,m,H-9), 3.99(3H,s,OMe), 4.05(3H,s,OMe), 4.48(1H,dd,J₁=7.5,J₂=3 Hz,H-8), 5.75(1H,s,OH-14), 6.80(1H,d,J=1.5 Hz,H-6), 7.01(1H,d,J=8 Hz,H-15), 7.10(1H,d,J=1.5 Hz,H-4), 7.30(1H,dd,J₁=8, J₂=2 Hz,H-16), 7.33(1H,d,J=2 Hz,H-12). ¹³C NMR(60 MHz): δ9.6(Me-3), 16.9(Me-10), 56.1, 56.2(2×OCH₃), 72.5(C-9), 80.1(C-8), 105.3(C-6), 109.5(C-12), 109.9(C-4), 110.1(C-3), 114.5(C-15), 120.7(C-16), 123.5(C-11), 133.0(C-3a), 136.4(C-5), 142.5(C-7a), 145.0(C-7), 145.9(C-14), 146.6(C-13), 152.6(C-2). EIMS m/z (rel.int.):358[M]⁺(100), 328(16), 314(21), 313(81), 285(52), 258(11), 257(57), 253(28), 225(14), 133(13).

### (2R,3R)-2,3-Dihydro-2-(4-hydroxy-3-methoxyphenyl)-3-hydroxymethyl-7-methoxy-5-(E)-propenylbenzofuran (Compound 19)

Amorphous (6 mg). TLC:R_{f}0.3(S-1); anisaldehyde:red. [α]_{D}+65° (c.0.2). CDλₘₐₓnmΔε):235(−3.15), 260(+3.14), 285(+2.39). IRνₘₐₓcm⁻¹:3543(OH), 3019, 1613, 1518, 1499, 1466. UVλₘₐₓnm(logε):204(4.59), 218(4.49), 273(4.23); +NaOH:211(4.93), 268(4.38). ¹H NMR(CD₃OD): δ1.78(3H,dd,J₁=6,J₂=2 Hz,Me10), 3.47(1H,m,H-3), 3.78(2H,d,J=7 Hz,CH₂OH), 3.80(3H,s,OMe), 3.86(3H,s,OMe), 5.50(1H,d,J-6 Hz,H-2), 6.11(1H,dq,J₁=16,J₂=6.5 Hz,H-9), 6.33(1H,dq,J₁=16,J₂=2 Hz,H-8), 6.76(1H,d,J=8 Hz,H-15), 6.82(1H,dd,J₁=8,J₂=2 Hz,H-16), 6.86(1H,br s,H-4), 6.88(1H,br s,H-6), 6.94(1H,d,J=2 Hz,H-12). ¹³C NMR(60 MHz):18.3(Me-10), 53.7(C-3), 56.0(2×OMe), 64.0(CH₂OH-3), 88.7(C-2), 108.8(C-12), 110.0(C-6), 113.9(C-4), 114.3(C-15), 119.4(C-16), 123.8(C-9), 127.9(C-11), 129.7(C-8), 132.3(C-5), 133.0(C-3a), 144.4(C-7), 145.7(C-14), 146.7(C-7a), 147.6(C-13), EIMS m/z (rel.int.):342[M]₊(52), 324(78), 310(20), 309(100), 293(28), 292(32), 221(10), 165(14), 152(13), 151(22), 137(17).

### erythro-1-(4-Acetoxy-3-methoxyphenyl)-2-[4-(7-methoxy-3-methyl-5-(E)-propenylbenzofuran-2-yl)-2-methoxyphenoxy]propylacetate (Compound 22)

Colourless crystals (5 mg). MP 156-158° (from MeOH). TLC:R_{f}0.54(S-3); anisaldehyde:grey. [α]_{D}+18° (c.0.1). IRνₘₐₓcm⁻¹:3018, 1762, 1741, 1510. UV λₘₐₓnm(logε):226(4.08), 266(4.10), 308(4.08). ¹H NMR: δ1.31(3H,d,J=6.5 Hz,Me-9′), 1.89(3H,dd,J₁=6.5,J₂=1.5 Hz,Me-10), 2.11(3H,s,MeCO-7′), 2.25(3H,s,MeCO-4′), 2.40(3H,s,Me-3), 3.83(3H,s,OMe), 3.89(3H,s,OMe), 4.01(3H,s,OMe), 4.77(1H,m,H-8′), 5.91(1H,d,J=4.5 Hz,H-7′), 6.24(1H,dq,J₁=16,J₂=6 Hz,H-9), 6.50(1H,dq,J₁=16,J₂=1.5 Hz,H-8), 6.80(1H,d,J=1.5 Hz,H-6), 6.91(1H,d,J=8 Hz,H-15), 6.96(1H,d,J=8.5 Hz,H-6′), 6.97(1H,dd,J₁=8.5, J₂=2 HzH-5′), 7.01(1H,br s,H-4), 7.08(1H,d,J=2 Hz,H-2′), 7.29(1H,dd,J₁=8,J₂=2 Hz,H-16), 7.32(1H,d,J=2 Hz,H-12). ¹³C NMR: δ9.6(Me-3), 15.5(Me-9′), 18.4(Me-10), 20.7(MeCO-4′), 21.2(MeCO-7′), 56.0,56.1(3×OMe), 76.6(C-7′), 78.0(C-8′), 104.7(C-6), 109.2(C-4), 110.8(C-3), 111.3(C-12), 112.1(C-2′), 117.7(C-5′), 119.6(C-15), 119.9(C-6′), 122.4(C-16), 124.4(C-9), 125.8(C-11), 131.5(C-8), 133.0(C-3a), 133.7(C-5), 135.9(C-1′), 139.6(C-14′), 142.2(C-7a), 144.9(C-7), 147.1(CO14), 150.9(C-13), 151.3(C-3′), 168.9(MeCO-4′), 169.9(MeCO-7′). EIMS m/z (rel.int.):588[M]₊(6), 366(14), 325(20), 324(100), 265(31), 223(54), 181(27), 164(25).

### threo-1-(4-Acetoxy-3-methoxyphenyl)-2-[4-(7-methoxy-3-methyl-5-(E)-propenylbenzofuran-2-yl)-2-methoxyphenoxy]propyl-acetate (Compound 23)

Mp 155-158° (frm MeOH). TLC:R_{f}0.54(S-3); anisaldehyde:grey. [α)_{D}+35° (c.0.1). IRνₘₐₓcm⁻¹:3018, 1762, 1741, 1510. UV λₘₐₓnm(logε):226(4.08), 266(4.10), 308(4.08). ¹H NMR: δ1.24(3H,d,J=6.5 Hz,Me-9′). 191(3H,dd,J₁=6.5, J₂=2 Hz,Me-10), 2.04(3H,s,MeCO-7′), 2.30(3H,s,MeCO-4′), 2.43(3H,s,Me-3), 3.85(3H,s,OMe), 3.92(3H,s,OMe), 4.04(3H,s,OMe), 4.65(1H,m,H-8′), 5.99(1H,d,J=6.5 Hz,H-7′), 6.22(1H,dq,J₁=16,J₂=6.5 Hz, H-9), 6.50(1H,dq,J₁=16,J₂=2 Hz,H-8), 6.84(1H,d,J=1.5 Hz,H-6), 6.99(1H,dd,J₁=8,J₂=2 Hz, H-16), 7.02(1H,d,J=8Hz,H-15), 7.03(1H,d,J=1.5 Hz,H-4), 7.03(1H,d,J=8.5 HzH-15′), 7.04(1H,d,J=2 Hz,H-12), 7.31(1H,dd,J₁=8.5,J₂=2 Hz,H-16′), 7.35(1H,d,J=2 Hz,H-12′), ¹³C NMR: δ9.6(Me-3), 16.7(Me-9′), 20.7(MeCO-4′), 21.1(MeCO-7′), 56.0,56.1(3×OMe), 76.6(C-7′), 77.8(C-8′), 104.6(C-6), 109.2(C-4), 110.7(C-3), 111.2(C-12), 111.9(C-2′), 116.8(C-5′), 119.8(C-15), 119.9(C-6′), 122.7(C-16), 124.4(C-9), 125.5(C-11), 131.5(C-8), 133.0(C-3a), 133.7(C-5), 136.0(C-1′), 139.8(C-4′), 142.2(C-7a), 144.9(C-7), 147.8(C-14), 150.5(C-2), 151.0(C-13), 151.2(C-3′), 168.8(MeCO-4′), 169.9(MeCO-7′). EIMS m/z (rel.int.):588[M]₊(6), 366(15), 325(20), 324(100), 265(30), 223(54), 181(27), 164(25).

### threo-1-[2-(4-Hydroxy-3-methoxyphenyl)-7-methody-3-methylbenzofuran-5-yl]-2-[4-(3-methyl-5-(e)-propenylbenzofuran-2-yl)-2-methoxyphenoxy]propan-1-ol (Compound 24)

Amorphous (3 mg). TLC:R_{f}0.69(S-2); anisaldehyde:violet. [α]_{D}+20° (c.0.2). IRνₘₐₓ cm⁻¹:3540 (OH), 3020, 2938, 1614, 1511, 1466. UVλₘₐₓnm(logε):229(4.16), 266(4.20), 308(4.14); +NaOH: 239(4.43), 330(4.46). ¹H NMR: δ0.98(3H,d,J=6.5 Hz,Me-10′), 1.89(3H,dd,J₁=6.5, J₂=1.5 Hz,Me-10), 2.36(3H,s,Me-3′), 2.43(3H,s,Me-3), 3.98, 4.01, 4.02, 4.06 (12H,s.4×Me), 4.22(1H,m,H-9′), 4.69(1H,d,J=8.5 Hz,H-8′), 5.74(1H,s,OH-14′), 6.20(1H,dq,J₁=16,J₂=6.5 Hz,H-9), 6.48(1H,dq,J₁=16,J₂=1.5 Hz-H-8), 6.81(1H,d,J=8.5 Hz,H-15), 6.81(1H,d,J=1.5 Hz,H-6), 6.89(1H,d, J=1.5 Hz,H-6′), 7.00(1H,d,J=8 Hz,H-15′), 7.01(1H,d,J=1.5 Hz,H-4), 7.07(1H,dd,J₁-8.5,J₂=2 Hz,H-16), 7.09(1H,d,J=1.5 Hz,H-4′), 7.29(1H,dd, J₁=8.5,J₂=2 Hz,H-16′), 7.31(1H,d,J=2 Hz,H-12′), 7.35(1H,d,J=2 Hz,H-12). ¹³C NMR: δ9.6(Me-3), 9.7(Me-3′), 18.0(Me-10′), 18.4(Me-10), 56.0, 56.1,56.3(4×OMe), 71.8(c-9′), 91.3(C-8′), 104.7(C-6), 105.4(C-6′), 109.2(C-4), 109.6(C-12′), 110.2(C-12), 110.4(c-4′), 110.6(C-3), 110.9(C-3′), 114.5(C-15), 118.5(C-15′), 119.8(C-16), 120.8(C-16′), 123.2(C-11′), 124.5(C-9), 126.2(C-11), n131.4(C-8), 132.9(C-31′), 133.1(C-3a), 133.7(C-5), 133.9(C-5′), 142.2(C-7a), 142.6(C-7a′), 144.9(C-7), 145.2(C-7′), 145.9(C-14′), 146.6(C-13′), 147.9(C-4), 150.7(C-13), 151.1(C-2′), 151.9(C-2). DCIMS m/z (rel.int.):665[m+h]⁺(10), 381(9), 367(12), 343(12), 342(25), 341(100), 340(24), 326(22), 325(94), 324(44).

### 2-Methoxy-4-[7-methoxy-3-methyl-5-(E)-propenylbenzofuran-2-yl]-6-[4-(7-methoxy-3-methyl-5-(E)-propenylbenzofuran-2-yl)-2-methoxyphenoxy]phenol (Compound 25)

Amorphous (9 mg). TLC:R_{f}0.83(S-1); anisaldehyde:grey. IRνₘₐₓcm⁻¹:3.538(OH), 3020, 2939, 1613, 1599, 1510. UVλₘₐₓnm(logε):233(4.14), 267(4.48), 309(4.46); +NaOH:215(5.23), 316(4.41). ¹H NMR(250 MHz): δ1.91(6H,m,Me-10 and Me10′), 2.31 (3H,s,Me-3′), 2.42(3H,s,Me-3), 3.99,4.01,4.04(12H,s,4×OMe), 6.20(2H,m,H-9 and H-9′), 6.47(1H,dq,J₁=16,J₂=1.5 Hz,H-8′), 6.50(1H,dq,J₁=16,₂=1.5 Hz,H-8), 6.81(1H,d,J=1.5 Hz,H6′), 6.84(1H,d, J=1.5 Hz,H-6), 7.00(1H,d,J=1.5 Hz,H-4′), 7.03(1H,d,J=1.5 Hz,H4), 7.04(1H,d,J=2 Hz,H-12′), 7.05(1H,d,J=8 Hz,H-15), 7.18(1H,d,J=2 Hz,H16′), 7.31(1H,dd,J₁=8,J₂=2 Hz,H-16), 7.45(1H,d,J=2 Hz,H12). ¹³CNMR(60 MHz): δ9.5,9.6(Me-3,Me-3′), 18.4(Me-10,Me-10′), 56.1,56.3,56.5(4×OMe), 104.7,104.8(C-6,C-6′), 106.1(C-16′), 109.3,109.8(C-4,C-4′), 111.2(C-3′), 111.3(C-12), 111.5(C-3), 111.9(C-12′), 116.8(C-15), 119.9(C-16), 122.8(C-11), 124.4,124.5(C-9,C-9′), 127.6(C-11′), 132.7,132.9(C-8,C-8′), 133.7,133.8(C-3a,C-3a′), 137.3(C-14′), 142,1,142.3(C-71,C-7a′), 143.8(C-14), 144.8,144.9(C-7,C-7′), 145.8(c-15′), 148.2(C-13,C-13′), 150.4, 150.9(C-2,C-2′). DCIMS m/z (rel.int.):647[M +H]⁺(100), 646(44), 473(18), 369(12), 341(26), 339(16), 326(11), 325(46), 324(23), 309(34), 308(13), 283(20), 113(19), 107(18), 105(12).

### 8.2′,9.3′-Tetrahydro-bis-eupomatenoid-7 (Compound 26)

Crystals (4 mg). Mp 175-179° (from MeOH). TLC:R_{f}0.26(S-1); anisaldehyde:grey-blue. [α]_{D}±0° (c.0.1). IRνₘₐₓcm⁻¹:3540(OH), 3020,1618,1465. UV λₘₐₓnm(logε) 217(5.03), 279(4.83), 297(sh 4.79), +NaOH: 261(4.76), 305(4.75), 327(sh 4.78). ¹H NMRδ1.05(3H,s,Me-3′), 1.31(3H,d,J=6.5 Hz,Me-10), 1.86(3H,dd,J₁=6.5,J₂=1.5 Hz,Me-10′), 2.37(3H,s,Me-3), 2.97(1H,dq,J₁=11,J₂=6.5 Hz,H-9), 3.50(3H,z,OMe-13′), 3.76(1H,d,J=11 Hz,H-8), 3.80(3H,s,OMe-7), 3.92(3H,s,OMe-13′), 4.00(3H,s,OMe-7′), 6.17(1H,dq, J₁=16,J₂=6.5 Hz,H-9′), 6.42(1H,dq,J₁=16,J₂=1.5 Hz,H-8′), 6.48(1H,s,H-6), 6.63(1H,d,J=8.5 Hz,H-15′), 6.75(1H,d,J=2 Hz,H-12′), 6.83(1H,dd, J₁=8.5, J₂=2 Hz,H-16′), 6.85(1H,d,J=1.5 Hz,H-4′), 6.90(1H,d,J=8 Hz,H-15), 6.94(1H,s,H-4), 6.98(1H,d,J=1.5 Hz,H-6′), 7.22(1H,dd,J₁=8,J₂=2 Hz,H-16), 7.33(1H,d,J=2 Hz,H-12). ¹³C NMR: δ9.6 (Me-3), 16.2(Me-10), 18.5(Me-10′), 22.1(Me-3′), 42.7(C-9), 56.2(C-3′), 56.5,56.7,57.3,58.2(4×OMe), 98.1(C-8), 107.9(C-2′), 109.7(C-6), 110.6(C-4), 110.9(C-6′), 111.3(C-12), 111.8(C-4′), 115.5(C-15′), 116.5(C-15), 117.4(C-12′), 120.7(C-16′), 121.2(C-16), 124.1(C-9′), 124.5(C-11), 128.8(C-11′), 132.4(C-8′), 133.7(C-5′), 134.0(C-3a), 135.3(C-5), 136.0(C-3a′), 142.5(C-7a), 146.0(C-7), 146.4(C-7′), 146.9(C-13′), 147.7(C-7a′), 148.0(C-14′), 149.2(C-14), 152.6(C-2). CIMS m/z (rel.int.):649[M+H]⁺ (13), 648(7), 367(12), 326(25), 325(100), 324(88).

### 15-(Aristolactam-I-9-yl)-eupomatenoid-7 (Compound 27)

Yellow crystals (4 mg). Mp 165-170° (from MeOH). TLC:R_{f}0.43(S-2); anisaldehyde:green. IR νₘₐₓ cm⁻¹: 3531,3442,3020,3011,1699,1610,1482,1466. UV λₘₐₓnm(logε):256 (4.83), 267(sh 4.79), 301(4.73), 405(4.00). ¹H NMR (C₅D₅N): δ1.86 (3H,dd,J₁=6.5,J₂=1.5 Hz,Me-10), 2.44(3H,s,Me-3), 3.52(3H,s,OMe-8′), 3.80(3H,s,OMe-13), 3.96(3H,s,OMe-7), 6.30(1H,dq,J₁=16,J₂=6.5 Hz,H-9), 6.34(2H,d,J=1 Hz,OCH₂O), 6.63(1H,dq,J₁=16,J₂=1.5 Hz,H-8), 7.09(1H,d,J=1.5 Hz,H-6), 7.13(1H,dd,J₁=8,J₂=1 Hz,H-7′), 7.27(1H,d,J=1.5 Hz,H-4), 7.57(1H,d,J=2 Hz,H-12), 7.58(1H,t,J=8 Hz,H-6′), 7.81(1H,d,J=2 Hz,H-16), 7.84(1H,s,H-2′), 8.57(1H,dd,J₁=8,J₂=1 Hz,H-5′), 11.26(1H, br s,OH), 12.02(1H,br s, NH). ¹³C NMR(C₅D₅N): δ9.8(Me-3), 18.5(C-10), 55.9,56.4,56.5(3×OCH₃), 103.4(OCH₂O), 105.1(c-6), 106.0(C-2′), 109.6,, 109.7(C-4,C-12), 111.5(C-7′), 112.6(C-4a′), 113.2(C-9′), 121.0(C-1′), 121.8(C-5′), 122.4(C-16), 124.4(C-9), 125.6(C-4b′), 126.1(C-6′), 127.9(C-11), 129.0(C-15), 132.3)C-8), 133.8(C-3a), 134.3(C-5), 136.1(C-10′), 142.6(C-7a), 145.6(C-7), 146.6(C-14), 1.47.7(C-4′), 148.6(C-13), 149.0(C-3′), 152.2(C-2), 158.8(C-8′), 169.7(CO). EIMS m/z (rel.int.): 615[M]⁺(100), 584(12), 583(11), 308(25), 292(14), 285(10).

### 14-O-α-Cadinyl-eupomatenoid-7 (Compound 28)

Oil (3.5 mg). TLC:R_{f}0.78(S-1); anisaldehyde:grey. [α]_{D}+39°(C.0.3). IRνₘₐₓcm⁻¹:3019,2917,1614,1599,1505,1481,1450. UV λₘₐₓnm(logε): 235(4.45), 265(4.48), 311(4.39). ¹H NMR: δ0.77(3H,d,J=7 Hz,Me-13′ or Me-14′), 0.90(3H,d,J=7 Hz,Me-13′ or Me-14′), 1.25(3H,s,Me-15′), 1.71(3H,s,Me-11′), 1.92(3H,dd,J₁=6.5,J₂=1.5 Hz,Me-10), 2.43(3H,s,Me-3), 3.90(3H,s,OMe-13), 4.04(3H,s,OMe-7), 5.53(1H,br s,H-4′), 6.22(1H,dq,J₁=16,J₂=6.5 Hz,H-9), 6.51(1H,dq,J₁=16,J₂=1.5 Hz,H-8), 6.83(1H,d,J=2 Hz,H-6), 7.04(1H,d,J=8Hz,H-15), 7.05(1H,d,J=2 Hz,H-4), 7.26(1H,dd,J₁=8,J₂=2 Hz,H-12), 7.32(1H,d,J=2 Hz,H-16). ¹³C NMR: δ9.7(Me-3), 15.1(Me-13′), 18.4(Me-10), 18.5(Me-15′), 21.5(Me-14@), 21.9(C-9′), 23.1(C-1′), 23.9(Me-11′), 25.9(C-12′), 31.0(C-2′), 37.7(C-8′), 40.2(C-5′), 46.3(C-6′), 48.0(C-10′), 55.8,56.1(2×OMe), 84.9(C-9′), 104.7(C-6), 109.2(C-4), 110.9(C-12), 111.8(C-3), 119.2(C-16), 122.4(C-4′), 124.4(C-9), 125.8(C-15), 127.1(C-11), 131.5(C-8), 133.1(C-3a), 133.7(C-5), 135.2(C-3′), 142.3(C-7a), 144.9(C-14), 151.4(C-2), 154,5(C-13). DCIMS m/z (rel.int.):529[M+H]⁺(41), 528(14), 367(16), 326(11), 325(51), 324(100), 206(15), 205(93), 203(6).

### (2R,4S)-2-Hydroxy-6-methoxy-4,7-dimethyl-1-tetralone (Compound 34)

Amorphous (17 mg). TLC:R_{f}0.44(S-1); anisaldehyde:yellow. [α]_{D}−39°(c.01). CDλₘₐₓnm(Δε):220(+3.74), 270(−2.09), 295(+3.52), 315(−143). IRνₘₐₓcm⁻¹:3496(OH), 3016, 1674,(CO), 1609, 1497. UV λₘₐₓnm(logε):225(4.12), 274(4.09). ¹H NMR(250 MHz): δ1.46(3H,d, J=6.5 Hz,Me-e), 176(1H,ddd,J₁=13.5,J₂=J₃=12.5 Hz,H-3ₐₓ), 2.21(3H,br s,Me-7), 2.48(1H,ddd,J₁=12.5,J₂=J₃=5.5 Hz,H-3_{eq}), 3.16(1H,m,H-4), 3.91(3H,s,OMe), 4.33(1H,dd,J₁=13.5, J₂=5.5 Hz,H-2ₐₓ), 6.77(1H,br s,H-5), 7.84(1H,br d,J=1 Hz,H-8). ¹³C NMR(60 MHz): δ15.7(Me-7), 20.5(Me-4), 31.6(C-4), 40.8(C-3), 55.5(OMe), 73.0(C-2), 107.0(C-5), 122.8(C-8a), 126.1(C-7), 129.7(C-8), 149.0(C-4a), 162.9(C-6), 198.5(CO). EIMS m/z (rel.int.):220[M]⁺(68), 202(25), 177(19), 176(100), 175(61), 174(31), 159(12), 148(33), 147(14), 133(37), 117(14), 115(13).

### EXAMPLE 2

Determination of Mutagenic and Antimutagenic Activity

The four major constituents of the benzene extract from Aristolochia taliscana roots—eupomatenoid-7 (7), eupomatenoid-1 (8), eupomatenoid-8 (17), Licarin-A (16)—were tested for their mutagenic and antimutagenic properties using the Ames bio-assay (Maron, D. M. and Ames, B. N., Mutation reasearch, 1983, 113, 173). The test compounds have the following structural formula:

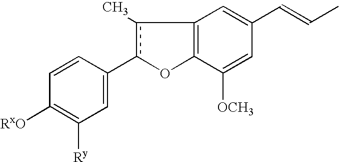

Eupomatenoid-1: R^{x} & R^{y}=OCH₂O, dotted line=double bond

Eupomatenoid-7: R^{x}=OH, R^{y}=OCH₃, dotted line=double bond

Eupomatenoid-8: R^{x} & R^{y}=OCH₂O, dotted line=single bond

Licarin-A: R^{x}=OH, R^{y}=OCH₃, dotted line=single bond

Method

Salmonella typhimurium strain TA 100 was used as the test organism and 2-amino-anthracene (2-AA) and 2-nitrofluorene (2-NF) as standard mutagens, of which 1 μg were added to each test plate. In the experiments with 2-AA, “S9 Mix” (derived from phenobarbital treated rat liver cells (De Flora, S., Camoirana, A., D'Agostini, F. and Balansky, R., Mutation Research, 1992, 267, 183) was also added.

Results

None of the tested substances showed any mutagenic activity. Eupomatenoid-7 (7) exhibited strong antimutagenic effects against 2-aminoanthracene as well as against 2-nitrofluorene (Tab. 4). Licarin-A (16) and eupomatenoid-1 (8) were found to be antimutagenically active only in the experiment against 2-AA but not against 2-NF (Tab. 5). However, eupomatenoid-8 (17) did not show any antimutagenic effect in the test systems used (Tab. 6).

Eupomatenoid-7 (7)

TABLE 4
Results from the experiments on antimutagenic
activity of eupomatenoid-7 (7).

| | Residual mutagenic activity (%) | |
|---|---|---|
| | observed for: | |
| Amount of compound added [μg) | 2-AA | 2-NF |
| 50 | 4 | 16 |
| 100 | 0 | 0 |

(±)-Licarin-A (6)

TABLE 5
Results from the experiments on antimutagenic
activity of (±)-licarin-A (6)

| | Residual mutagenic activity (%) | |
|---|---|---|
| | observed for: | |
| Amount of compound added [μg) | 2-AA | 2-NF |
| 50 | 31 | 94 |
| 100 | 6 | 85 |

Eupomatenoid-1 (8)

TABLE 6
Results from the experiments on antimutagenic
activity of eupomatenoid-1 (8)

| | Residual mutagenic activity (%) | |
|---|---|---|
| | observed for | |
| Amount of compound added [μg) | 2-AA | 2-NF |
| 50 | 49 | 99 |
| 100 | 44 | 93 |

Eupomatenoid-8 (17)

TABLE 7
Results from the experiments on antimutagenic
activity of eupomatenoid-8 (17).

| | Residual mutagenic activity (%) | |
|---|---|---|
| | observed for | |
| Amount of compound added [μg) | 2-AA | 2-NF |
| 50 | 90 | 100 |
| 100 | 73 | 95 |

### EXAMPLE 4

Cytotoxicity Studies

The toxicity of compounds isolated from Aristolochia Taliscana was assayed using the well known brine shrimp bioassay. The cytotoxicities of compounds of the invention, expressed as percentage “death rates” after 24 hours, at varying concentrations, are shown in Table 8 below.

TABLE 8
Cytotoxicities of Compounds in the Brine Shrimp Assay

| | “Death Rate” After | | | |
|---|---|---|---|---|
| | 24 Hours (%) | | | |
| SUBSTANCE | 10 ppm | 100 ppm | 500 ppm | LC₅₀ (ppm) |
| Aristolactam B (3) | 5 | 9 | 29 | >500 |
| Aristolactam C (4) | 0 | 0 | 3 | >500 |
| Eupomatenoid-7 (7) | 27 | 38 | 38 | >500 |
| Eupomatenoid-1 (8) | 12 | 16 | 20 | >500 |
| Licarin-A (16) | 93 | 93 | 96 | <10 |
| Eupomatenoid-8 (17) | 9 | 27 | 42 | >500 |
| Dihydrocarinatidine (21) | 26 | 53 | 80 | ca. 120 |
| Coniferyl alcohol (29) | 0 | 0 | 15 | >500 |
| Vanillin (31) | 5 | 0 | 12 | >500 |
| Compound 34 | 52 | 86 | 100 | <10 |
| E-Germacrene D (38) | 0 | 39 | 100 | ca. 126 |
| Podophyllotoxin | 74 | 93 | 100 | <10 |

### EXAMPLE 5

Antifungal Activity

The antifungal activities of compounds of the invention was determined using a plate diffusion method. Plates containing medium and a fungal species were made up and 150 microgramme aliquots of a test compound of the invention were spotted onto the plate. The diameter of inhibition of fungal growth around the test compound was then determined. The results of the tests are shown in Table 9 below.

TABLE 9
Antifungal Activity

| | Test Microorganism | | |
|---|---|---|---|
| | Botryis | Rhizoctonia | Saprolegnia |
| COMPOUND | cinerea | solani | asterophora |
| Aristolactam B (3) | − | + | − |
| Aristolactam C (4) | + | + | ++ |
| Eupomatenoid-7 (7) | − | − | − |
| Eupomatenoid-1 (8) | − | − | − |
| Licarin-A (16) | − | ++ | − |
| Eupomatenoid-8 (17) | − | − | − |
| Dihydrocarinatidine (21) | + | + | + |
| Coniferyl alcohol (29) | − | − | − |
| Vanillin (31) | − | − | − |
| Compound 34 | ++ | ++ | ++ |
| E-Germacrene D (38) | + | + | − |

− = no inhibition
+ = 5 mm diameter inhibition
++ = 5-10 mm diameter inhibition

### EXAMPLE 6

Use of Aristolochia taliscana Extracts in the Treatment and Management of Aids

An aqueous alcoholic extract was prepared by extracting roots from Aristolochia taliscana with aqueous ethanol and concentrating the resulting solution to 65% solids content by evaporation under reduced pressure. Ethanol was then added to the solution to give a concentration equivalent to 1 litre of solution for every kilogramme of raw material. The result was a brown liquid which was administered without further purification.

Case Study 1

Patient I, whose identity cannot be revealed for medical confidentiality reasons, had been diagnosed as suffering from AIDS, and had previously been treated with azidothymidine (AZT), dideoxycytidine (DDC) and dideoxyinosine (DDI) but had been forced to discontinue the treatment because of the side effects. When initially examiner prior to entry into the present study, he was suffering from a low CD4 count, gastrointestinal disturbances, a severe scalp infection and weight loss. Patient I was treated by daily oral administration of several drops of the alcoholic extract of Aristolochia taliscana. After forty five days, the gastrointestinal problems had disappeared, the scalp infection had gone, and he had gained seven kilogrammes in weight.

Case Study 2

Patient number II, a resident of Mexico City, and who had been diagnosed as being HIV positive, was treated by daily oral administration of the alcoholic extract of Aristolochia taliscana over a period of nearly five years. At the end of that period, Patient II's CD4 count was approximately 60. When the CD4 count falls below about 200, the immune system is generally unable to cope with infection of bacterial or fungal origin and patients with such a reduced immune function typically die from infections of one kind or another. It owuld therefore have been expected that Patient II, having such a low CD4 count, would have succumbed to infection during the five year period. However, despite the low CD4 count, Patient II remained healthy and active and free from the symptons of AIDS during the period of treatment. It would therefore appear that the Aristolochia taliscana extract does not function by stimulating the immune sytem, but by some other, at present unknown, mechanism.

1. The use of an extract from an Aristolochia species or one or more compounds isolable therefrom, for the manufacture of a medicament for the treatment of AIDS.

2. The use of an extract from an Aristolochia species or one or more compounds isolable therefrom for the manufacture of a medicament for preventing or reversing cachexia in AIDS patients.

3. The use according to claim 1 or claim 2 wherein the Aristolochia species is Aristolochia taliscana.

4. The use according to any one of claims 1 to 3 wherein the composition contains at least 10%, preferably at least 20%, and more preferably at least 25% by weight of a phenylbenzfuran.

5. The use according to claim 4 wherein the phenylbenzfuran is a eupomatenoid.

6. The use according to claim 4 or claim 5 wherein the phenylbenzfuran contains a phenolic group.

7. The use according to claim 6 wherein the phenylbenzfuran is eupomatenoid-7.

8. The use according to any one of the preceding claims wherein the composition contains Licarin-A.

9. The use according to any one of the preceding claims wherein the composition contains a cytotoxic tetralone compound.

10. The use according to any one of the preceding claims wherein the composition contains a 2-hydroxy-1-tetralone compound.

11. The use according to claim 9 or claim 10 wherein the tetralone compound compound is (2R,4S)-2-Hydroxy-6-methoxy-4,7-dimethyl-1-tetralone.

12. The use according to any one of the preceding claims wherein the composition contains at least 25% by weight of a phenolic eupomatenoid compound (such as eupomatenoid-7), at least 8% of Licarin-A and at least 8% of a non-phenolic eupomatenoid compound (such as eupomatenoid-8).

13. The use according to any one of the preceding claims wherein the composition contains an aristolactam.

14. The use according to any one of the preceding claims wherein the extract has been prepared by extraction of plant material from the Aristolochia species with an organic solvent.

15. The use according to claim 15 wherein the organic solvent is an alcoholic solvent such as ethanol or methanol or a mixture thereof.

16. The use according to claim 15 wherein the organic solvent is benzene, the solvent having been removed from the extract prior to use.

17. A method of treating Acquired Immune Deficiency Syndrome (AIDS) in a patient suffering from AIDS, which method comprises administering to the patient an effective treatment amount of an extract from an Aristolochia species or one or more anti-AIDS active compounds isolable therefrom, as defined in any one of the preceding claims.

18. A method of treating one or more symptoms associated with AIDS in a patient suffering therefrom, which method comprises administering to the patient an effective treatment amount of a compound or composition as defined in any one of claims 1 to 16.

19. A method of preventing or reversing cachexia, for example in AIDS patients or in patients suffering from a neoplastic disease such as a cancer, which method comprises administering to the patient an effective treatment amount of an extract from an Aristolochia species or one or more compounds isolable therefrom, as defined in any one of the preceding claims 1 to 16.

20. The use of a compound for the manufacture of a medicament for use in the treatment or alleviation of AIDS or the symptoms thereof, or the alleviation or reversal of cachexia, the compound being of the formula (I): 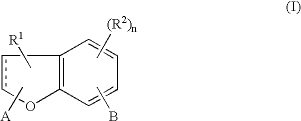
wherein the dotted line signifies a single or double bond; n is 0, 1, 2 or 3; A is a monocyclic aryl ring optionally substituted by one or more substituent groups which may be the same or different and are selected from R³O, R³, R³S, halogen; aryl and heteroaryl, wherein R³ is hydrogen, or a hydrocarbyl group optionally substituted by a hydroxy or hydrocarbyloxy group; B is selected from carboxy, carboxaldehyde, hydrocarbyl and hydrocarbyloxy groups wherein the hydrocarbyl group is acyclic or cyclic, and optionally contains one or more heteroatoms, and is optionally substituted by one or more hydroxy, alkoxy, alkenyloxy, alkynyloxy, aryloxy, aldehyde, alkanoyl, acetal, hemiacetal and carboxy groups; R¹ is hydrogen or a hydrocarbyl group optionally including one or more heteroatoms and optionally substituted by one or more substituents selected from hydroxy, hydrocarbyloxy and aryl groups; and R² is hydroxy or a hydrocarbyl or hydrocarbyloxy group optionally substituted by one or more substituents selected from hydroxy, hydrocarbyloxy and aryl groups.

21. The use according to claim 20 wherein the monocyclic aryl ring A is attached to the 2-position of the furan ring.

22. The use according to claim 20 or claim 21 wherein the aryl ring is a phenyl group.

23. The use according to any one of claims 20 to 22 wherein the group B is attached to the 5-position of the benzofuran group.

24. The use according to any one of claims 20 to 23 wherein there is only one group R².

25. The use according to claim 24 wherein the group R² is attached to the 7-position of the benzofuran ring.

26. The use according to any one of claims 21 to 25 wherein the dotted line signifies a double bond.

27. The use according to claim 21 wherein the compound of the formula (I) has the formula (II): 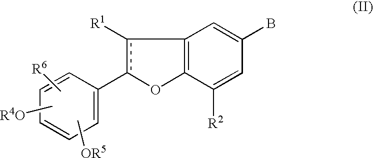
wherein B, R¹ and R² are as defined in any one of claims 6 to 11, R⁴ and R⁵ are the same or different and each is selected from hydrogen, C₁₋₂₀ hydrocarbyl, C₅₋₂₀ aryl, or C₅₋₂₀ oxygen-containing heteroaryl; R₆ is selected from C₁₋₂₀ hydrocarbyl or C₁₋₂₀ hydrocarbyloxy optionally substituted by one or more hydroxy, alkoxy, aralkyloxy groups; or R⁶ is C₅₋₂₅ aryl or oxygen or nitrogen-containing heteroaryl.

28. The use according to claim 27 wherein B is C₁₋₆ alkyl or alkenyl optionally substituted by one or more substituents selected from hydroxy, CHO, or R⁷O wherein R⁷ is a C₁₋₆ alkyl or alkenyl group.

29. The use according to claim 28 wherein the group B is selected from CH═CHCH₃, CH₂CH═CH₂, CH(OH)CH═CH₂, CH═CHCHO, CHO, CH═CHCH₂OH and CH(OH)CH(OH)CH₃.

30. The use according to claim 29 wherein B is CH═CHCH₃.

31. The use according to any one of claims 21 to 31 wherein R⁴ and R⁵ are selected from hydrogen, or C₁₋₆ alkyl, or R⁴ and R⁵ together define an alkylene group such as —CH₂—.

32. The use according to claim 31 wherein at least one of R⁴ and R⁵ is hydrogen.

33. The use according to any one of claims 27 to 32 wherein R⁶ is selected from hydrogen, halogen, C₁₋₆ alkoxy (e.g. methoxy), a 2-benzofuranyl ring, and an aristolactam group.

34. The use according to any one of claims 21 to 33 wherein each hydrocarbyl group is selected from aliphatic, alicyclic and aromatic groups.

35. The use according to claim 34 wherein the hydrocarbyl group is selected from alkyl, alkenyl, alkynyl, cycloalkyl, cycloalkylalkyl, cycloalkylalkenyl, cycloalkylalkynyl, aryl, aralkyl, aralkenyl, aralkynyl, optionally interrupted by one or more heteroatoms such as oxygen and sulphur.

36. The use according to claim 35 wherein the hydrocarbyl group is a C₁₋₆ alkyl group selected from methyl, ethyl, propyl, isopropyl, butyl, isobutyl and t-butyl; a cycloalkyl group selected from cyclopropyl, cyclobutyl, cyclopentyl, cyclohexyl, cycloheptyl, cyclooctyl, bicycloheptanyl, decalinyl, adamantyl, norbornyl and bicyclooctyl; an alkenyl or alkynyl groups selected from vinyl, ethynyl, allyl, 1-propenyl, propargyl, but-1-enyl, but-2-enyl, but-3-enyl and 3-methylbutenyl; a cycloalkenyl group selected from cyclopentenyl, cyclohexenyl and cycloheptenyl; an aryl groups selected from phenyl and naphthyl; or a phenylalkyl or phenylalkenyl groups selected from benzyl, phenethyl, phenylpropyl, phenylbutyl and styryl groups.

37. A compound of the formula (I) or (II) as defined in any one of the preceding claims for use in the treatment or alleviation of AIDS or the symptoms thereof, or the alleviation or reversal of cachexia; but provided that when R¹ is 3-methyl, R² is a single methoxy group at the 7-position, and either (i) the furan ring is unsaturated and is substituted at the 2-position with a 4-hydroxy-3-methoxyphenyl group or a 3,4-methylenedioxyphenyl group; or (ii) the furan ring is a 2,3-dihydrofuran ring and is substituted at the 2-position with a 4-hydroxy-3-methoxyphenyl group, then B is other than a prop-1-enyl group attached to the 5-position of the benzfuran ring.

38. A use, or a method of treatment as defined in any one of the preceding claims, wherein the compound is selected from the group consisting of:
(±)-5-(1-Hydroxyallyl)-2-(4-hydroxy-3-methoxyphenyl)-7-methoxy-3-methylbenzofuran;
2-(4-Hydroxy-3-methoxyphenyl)-3-hydroxymethyl-7-methoxy-5-(E)-propenylbenzofuran;
2-(4-Hydroxy-3-methoxyphenyl)-7-methoxy-3-methyl-5-[(E)-3-oxopropenyl]benzofuran;
5-Formyl-3-(4-hydroxy-3-methoxyphenyl)-7-methoxy-3-methylbenzofuran;
2-(4-Hydroxy-2-methoxyphenyl)-5-[(E)-3-hydroxypropenyl]-7-methoxy-3-methylbenzofuran;
2-(3,4-Dihydroxyphenyl)-7-methoxy-3-methyl-5-(E)-propenylbenzofuran;
erythro-5-(1,2-Dihydroxypropyl)-2-(4-hydroxy-3-methoxyphenyl)-7-methoxy-3-methylbenzofuran;
(2R,3R)-2,3-Dihydro-2-(4-hydroxy-3-methoxyphenyl)-3-hydroxymethyl-7-methoxy-5-(E)-propenylbenzofuran;
erythro-1-(4-Acetoxy-3-methoxyphenyl)-2-[4-(7-methoxy-3-methyl-5-(E)-propenylbenzofuran-2-yl)-2-methoxyphenoxy]propylacetate;
threo-1-(4Acetoxy-3-methoxyphenyl)-2-[4-(7-methoxy-3-methyl-5-(E)-propenylbenzofuran-2-yl)-2-methoxyphenoxy]propyl-acetate;
threo-1-[2-(4-Hydroxy-3-methoxyphenyl)-7-methoxy-3-methylbenzofuran-5-yl]-2-[4-(3-methyl-5-(e)-propenylbenzofuran-2-yl)-2-methoxyphenoxy]propan-1-ol;
2-Methoxy-4-[7-methoxy-3-methyl-5-(E)-propenylbenzofuran-2-yl]-6-[4-(7-methoxy-3-methyl-5-(E)-propenylbenzofuran-2-yl)-2-methoxyphenoxy]phenol;
8.2′, 9.3′-Tetrahydro-bis-eupomatenoid-7;
15-(Aristolactam-1-9-yl)-eupomatenoid-7;
14-O-α-Cadinyl-eupomatenoid-7; and
(2R,4S)-2-Hydroxy-6-methoxy-4,7-dimethyl-1-tetralone.
